(19) United States
(12) Patent Application Publication
(10) Pub. No.: US 2016/0116448 A1
(43) Pub. Date: Apr. 28, 2016

(54) pH-MODULATED QUENCHERS OF FLUORESCENCE

(71) Applicant: Berry & Associates, Inc., Dexter, MI (US)

(72) Inventors: John C. Hodges, Ann Arbor, MI (US); Richard H. Hutchings, Dexter, MI (US)

(21) Appl. No.: 14/870,624

(22) Filed: Sep. 30, 2015

Related U.S. Application Data
(60) Provisional application No. 62/057,757, filed on Sep. 30, 2014.

(51) Int. Cl. G01N 31/22; G01N 21/64; C07C 255/57; C07C 205/56; C07D 213/76

(52) U.S. Cl. CPC G01N 31/221; C07C 205/56; C07D 213/76; C07C 255/57; G01N 21/643; G01N 2021/6432; G01N 2021/6439

(57) ABSTRACT

The present disclosure relates to a compound of formula (I)

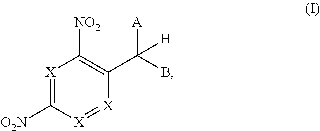

or a salt thereof, wherein variable A, B, and X are as defined herein. The disclosure also relates to covalent conjugates of formula (II). The disclosure further relates to the use of the compounds of formula (I) and covalent conjugates of formula (II) in methods for detecting a change in the pH of a mixture.

### CROSS REFERENCE TO RELATED APPLICATIONS

This application claims the benefit of U.S. Provisional Application No. 62/057,757, filed Sep. 30, 2015, the entire contents of which are incorporated by reference.

### BACKGROUND

The present disclosure relates to certain organic dyes and their use as pH-modulated quenchers of fluorescence.

Optical chemical transduction systems are based on chemical reagents that change their optical properties when they interact with an analyte of interest. See, e.g., Dorota Wencel, Tobias Abel, and Colette McDonagh; Anal. Chem. 2014, 86, 15-29. Fluorescence based optical chemical sensors utilize changes in fluorescence to transduce chemical information into optical signals. Fluorescence based optical chemical sensors and detectors have found a wide variety of applications in biology, chemistry, and engineering. See, e.g., Amanda Cobos Correa, Carsten Schultz, Small molecule-based FRET probes, Laboratory Techniques in Biochemistry and Molecular Biology, Volume 33, 2009, Pages 225-288; Thi Nhu Ngoc Van, May C. Morris, Fluorescent Sensors of Protein Kinases: From Basics to Biomedical Applications Progress in Molecular Biology and Translational Science, Volume 113, 2013, Pages 217-274. Fluorescence based optical transduction systems are particularly useful when compared to traditional absorption based systems due to their sensitivity.

One type of fluorescence-based optical chemical sensor utilizes two kinds of molecules, a fluorescence donor and a fluorescence quencher, to generate a signal. In such systems, the fluorescence donor generally is insensitive to the analyte. Chemical sensing results from analyte-induced changes in the quencher, which results in changes in fluorescence energy transfer efficiency from donor to the quencher. For example, a fluorescence-based sensor useful for the detection of CO₂ involves FRET quenching of a pH insensitive donor (sulforhodamine) with a pH sensitive quencher (m-cresol purple). See, e.g., Rao et. al., Biotechnol. Prog. 1996, 12, 266. Similarly, a lifetime-based optical NH₃ sensor based on the principle of fluorescence resonance energy transfer involves a sulforhodamine 101 donor, a pH sensitive bromocresol green quencher, an ethyl cellulose support, and a tri(butyl)phosphate plasticizer. Changes in the concentration of NH₃ cause changes in the decay time of the sulforhodamine 101, which is measured by phase-modulation fluorometry. See, e.g., Analytical Biochemistry 1995, 227, 309; Biotechnol. Prog. 1996, 12, 266.

Despite the emergence of the foregoing quenching systems, limitations in pH-sensitive quenching technology still exist.

### SUMMARY OF INVENTION

The present disclosure relates to a compound of formula (I)

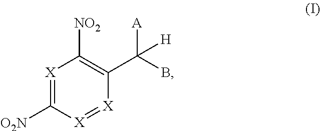

or a salt thereof, wherein A, B and X are as defined below.

The present disclosure also relates to a covalent conjugate of formula (II)

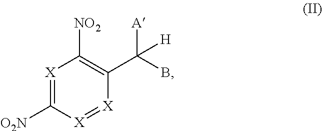

or a salt thereof, wherein A′, B and X are as defined below.

The present disclosure also relates to a method for detecting a change in the pH of a mixture comprising providing a mixture comprising a fluorescence donor and a compound of formula (I) or a salt thereof or a covalent conjugate of formula (I) or a salt thereof; irradiating the resulting mixture at a first time with light having a wavelength suitable to excite the fluorescence donor; measuring detectable fluorescence emitted by the fluorescence donor while irradiating the mixture at said first time; irradiating the resulting mixture at a second time with light having a wavelength suitable to excite the fluorescence donor; measuring detectable fluorescence emitted by the fluorescence donor while irradiating the mixture at said second time; and comparing the detectable fluorescence emitted by the fluorescence donor at said first time with the detectable fluorescence emitted by the fluorescence donor at said second time to detect a change in the pH of the mixture; wherein a decrease in detectable fluorescence at said second time relative to said first time indicates an increase in the pH of the mixture, and wherein an increase in detectable fluorescence at said second time relative to said first time indicates a decrease in the pH of the mixture.

### BRIEF DESCRIPTION OF THE DRAWINGS

FIG. 1 depicts the absorption spectrum of a compound of formula (I).

### DETAILED DESCRIPTION

As used herein, the following definitions shall apply unless otherwise indicated:

The term “fluorescence donor” refers to a molecule that emits light, typically in the visible and/or infrared region (i.e., having a wavelength between about 380 nm and about 1,000 nm), upon excitation by light of a shorter wavelength than the light emitted.

Examples of fluorescence donors include without limitation fluorescein, tetrachlororfluorescein (TET), hexachlorofluorescein (HEX), dichlorodimethoxyfluorescein (Joe) tetramethylrhodamine, Texas Red, Rhodamine-X (ROX), Cy3, Cy5, Cy5.5, and the like.

The term “salt thereof,” when referring to a compound of formula (I), means an ion pair consisting of an ion formally derived from the protonation or deprotonation of the compound of formula (I) and a counterion, whether existing in the solid state or in solution.

The term “alkyl” means a saturated hydrocarbon group that may be linear, branched, cyclic, or any combination thereof. In some embodiments, an alkyl group may have a specified number of carbon atoms. For example, a “C₁-C₆ alkyl” group is an alkyl group having between one and six carbon atoms. Exemplary alkyl groups include methyl, ethyl, n-propyl, isopropyl, n-butyl, t-butyl, isobutyl, sec-butyl, cyclohexylmethyl, 3,3,4,4-tetramethylcyclopent-1-yl, and the like.

The term “perfluoroalkyl” means an alkyl group in which all of the hydrogen atoms have been replaced with fluorine atoms.

The term “NHS” means N-hydroxysuccinimide. Accordingly, CH₃CO(NHS) is 2,5-dioxopyrrolidin-1-yl acetate, also known as the NHS ester of acetic acid. The (NHS) group refers to the following substituent: 0

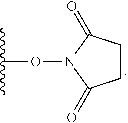 [SMILES: *ON1C(=O)CCC1=O]

The term “CEP” means 2-(cyanoethyl)-N,N-(di-2-propylamino)phosphityl.

The term “DMT” means bis(4-methoxyphenyl)phenylmethyl, also known as dimethoxytrityl. Accordingly, DMT-CI means dimethoxytrityl chloride.

The term “Conjugated Species,” when referring to a portion of a covalent conjugate, refers to a protein, antibody, peptide, nucleic acid, oligonucleotide, solid support, soluble polymer, insoluble polymer, dendrimer, saccharide, fatty acid, lipid, triglyceride, or phospholipid. The Conjugated Species is bound covalently to the remaining portion of the covalent conjugate at any chemically feasible location on the Conjugated Species.

The term “DNA” means deoxyribonucleic acid.

The term “RNA” means ribonucleic acid.

The term “nucleic acid” means a polymeric macromolecule made up of nucleotide monomers. Nucleic acids include DNA and RNA of natural and synthetic origin, including fragments of DNA and RNA that have been produced by enzymatic cleavage of larger DNA and RNA molecules and including DNA and RNA that have been chemically or enzymatically modified to provide chemical functionalities not typically found in nature.

The term “nucleotide” means the monomer subunit of a nucleic acid or an oligonucleotide. Nucleotides are composed of a heterocylic base, ribose or deoxyribose, and one or more phosphate groups. Heterocyclic bases incorporated in nucleotides include, without limitation, the natural purine and pyrimidine bases, adenine, cytosine, guanine, thymine, uracil, and hypoxanthine, post translationally modified natural bases, such as 5-hydroxymethylcytosine, 5-formylcytosine, 5-carboxycytosine, synthetic analogs of natural bases, such as pseudouracil, pseudocytosine, deazapurines, azapurines, deazapyrimidines, azapyrimidines, synthetic purines and pyrimidines with aldehyde, alkyne, amine, carboxy, hydroxyl anth thiol modifiers, synthetic purines and pyrimidines with cholesterol, psoralen, tocopherol, folate, and fluorophore labels, and the like.

The term “oligonucleotide” means a single-stranded polymer made up of between about 2 and about 250 nucleotides, preferably between about 6 and about 250 nucleotides.

The term “protein” means an organic polymer comprising one or more amino acid chains. Proteins include without limitation enzymes, globular proteins, fibrous proteins, membrane proteins, serum proteins, plasma proteins, and antibodies.

The term “peptide” means a natural or synthetic compound comprising a linear chain of between about 2 and about 30 amino acid residues linked by amide groups formed between the carboxyl group of one amino acid to the amino group of another.

The term “antibody” means a Y-shaped protein that, in biological systems, is secreted into the blood or lymph in response to an antigenic stimulus, such as a bacterium, virus, parasite, or transplanted organ, and that neutralizes the antigen by binding specifically to it.

The term “saccharide” means a molecule made up of one or more monosaccharide subunits, and includes monosaccharides, disaccharides, oligosaccharides, and polysaccharides.

The term “lipid” means any of a group of organic compounds, including the fats, oils, waxes, sterols, and triglycerides, that are insoluble in water but soluble in nonpolar organic solvents, are oily to the touch, and constitutes a principal structural material of living cells.

The term “phospholipid” means any of the various phosphorus-containing lipids, such as lecithin and cephalin, that are composed mainly of fatty acids, a phosphate group, and a simple organic molecule that are common in living organisms.

The term “triglyceride” means an ester of three fatty acids and glycerol.

The term “fatty acid” means any of a large group of monoprotic acids, especially those found in animal and vegetable fats and oils, having the general formula CₙH₂ₙ₊₁COOH.

The term “soluble polymer” means any of numerous natural and synthetic organic molecules comprised of one or more repeating units (monomers) which is of a low enough molecular weight to remain soluble in aqueous or organic solvent. For example linear polystyrene can be made in a form that remains soluble in many organic solvents such as dichloromethane, ethyl acetate, DMF, THF and the like, and linear polyethylene glycol can be made in a form that remains soluble in water and mixtures of water and water-miscible solvents such as acetonitrile, acetone, DMF, dimethylsulfoxide, and the like.

The term “insoluble polymer” means any of numerous natural and synthetic organic molecules comprised of one or more repeating units (monomers), which is of a sufficient molecular weight and/or rigid character to become insoluble. Some insoluble polymers swell when placed in a solvent but do not dissolve and others do not swell.

Examples of insoluble polymers include polystyrene-divinylbenzene, polyacrylates, polypropylene, Teflon, and the like.

The term “dendrimer” means any of numerous soluble or insoluble polymers that are highly branched and typically originate either from a single central atom or a central ring of atoms. The number of branches in a dendrimer typically increases in geometric fashion as the molecular weight rises.

The term “solid support” refers to a solid and insoluble material to which an organic compound or reagent may be attached during a synthetic chemical transformation or biological assay. Examples of solid support include, without limitation, polystyrene-divinylbenzene, CPG, paper, and solid surfaces.

The term “CPG” means controlled pore glass, which is a glass that has been made in a fashion so as to have a high surface area by virtue of many pores of a specific size. CPG is typically manufactured in small particles to further enhance the surface area. CPG is a common solid support that is used in oligonucleotide synthesis.

The term “solid surface” means the outer most boundary of a material. The interior walls of the well of a microtitre plate, the interior walls of test tubes, the exterior of gold particles, and the like are examples of solid surfaces that are applicable to chemical and biochemical assays.

The term “providing a mixture,” when referring to a mixture of two or more components, includes any method of obtaining a mixture of the two or more components, including obtaining the mixture from a third party or preparing the mixture.

The term “mixture,” when referring a mixture comprising two or more specified components, includes without limitation a solution, suspension, emulsion, or bi- or multi-phasic mixture comprising the two or more specified components. A mixture of two or more specified components may include one or more additional, unspecified components, such as solvents, reagents, and the like.

The term “irradiating,” when referring to a mixture of a fluorescence donor and a compound of formula (I), means exposing the mixture to light.

The term “light,” when referring to the light used to irradiate a mixture of a fluorescence donor and a compound of formula (I), means electromagnetic radiation, including without limitation ultraviolet light, visible light, and infrared light.

The term “detectable fluorescence,” when referring to fluorescence emitted by a fluorescence donor in a mixture of a fluorescence donor and a compound of formula (I), means electromagnetic radiation that can be measured by a fluorimeter.

The term “DCM” means dichloromethane.

The term “DIC” means diisopropylmethanediimine, also known as N,N′-d isopropyl-carbodiimide.

The term “DMAP” means N,N-dimethylpyridin-4-amine, also known as 4-dimethylaminopyridine.

The term “DMF” means N,N-dimethylformamide.

The term “lcaa-CPG” means long chain aminoalkyl linker on controlled pore glass beads, a common solid support used in automated oligonucleotide synthesis.

The term “NMI” means 1-methylimidazole, also known as N-methylimidazole.

The term “PYBOP” means (benzotriazol-1-yloxy)tripyrrolidinophosphonium hexafluorophosphate.

The term “MPLC” means medium pressure liquid chromatography.

The term “TEA” means triethylamine.

### Compounds of Formula (I)

In one aspect, the present disclosure relates to a compound of formula (I)

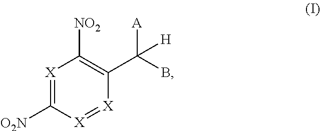

or a salt thereof, wherein:

A is CO₂R¹, CO₂R², C(O)NR³R⁴, CN, S(O)R⁵, SO₂R⁵, C(O)R⁶, C(O)R⁷, or CO(NHS);

B is selected from the group consisting of B¹, B², B³, and B⁴; wherein

B¹ is

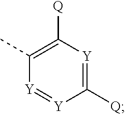

B² is selected from the group consisting of

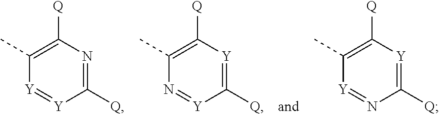

B³ is selected from the group consisting of

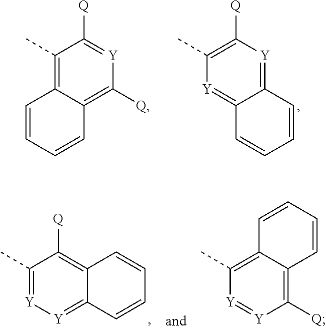

and

B⁴ is selected from the group consisting of

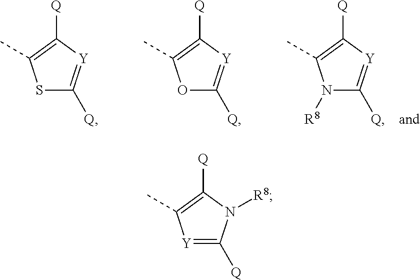

each X is independently N, C—H, C—Z¹, or C—Z²;

each Q is independently H, NO₂, Cl, Br, F, I, CH₃, OCH₃, CN, CO₂R⁹, C(O)NR¹⁰R¹¹, SO₂R¹², SOR¹², CF₃ or N₃;

R¹ is H or (C₁-C₁₂)alkyl;

R² is CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂OCH₂C≡CH, (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);

R³ is H, (C₁-C₁₂)alkyl, CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂OCH₂C≡CH, (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);

R⁴ is H, (C₁-C₆)alkyl, CH₂CH₂(C₄-C₁₂)perfluoroalkyl, or (CH₂CH₂O)ₙCH₂CH₂O(C₁-C₄)alkyl, or R³ and R⁴ together with the nitrogen atom to which they are attached form a ring selected from the group consisting of

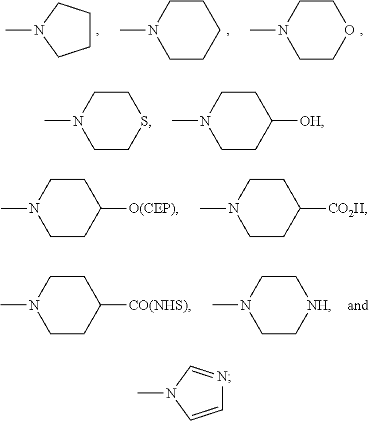

R⁵ is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;

R⁶ is H, (C₁-C₆)alkyl, or phenyl;

R⁷ is phenyl, wherein said phenyl is substituted with one to five substituents selected from the group consisting of CH₃, F, Cl, Br, I, OCH₃, OH, NO₂, CN, CF₃, N₃, and N(CH₃)₂;

R⁸ is H, (C₁-C₆)alkyl, CH₂CH₂(C₄-C₁₂)perfluoroalkyl), or (CH₂CH₂O)ₙCH₂CH₂O(C₁-C₄)alkyl,

R⁹ is H, CH₂CH₂(C₄-C₁₂)perfluoroalkyl), or (CH₂CH₂O)ₙCH₂CH₂O(C₁-C₄)alkyl;

R¹⁰ is H, (C₁-C₁₂)alkyl, CH₂CH₂(C₄-C₁₂)perfluoroalkyl), or (CH₂CH₂O)ₙCH₂CH₂O(C₁-C₄)alkyl;

R¹¹ is H, (C₁-C₆)alkyl, or (CH₂CH₂O)ₙCH₂CH₂O(C₁-C₄)alkyl, or R¹⁰ and R¹¹ together with the nitrogen atom to which they are attached form a ring selected from the group consisting of

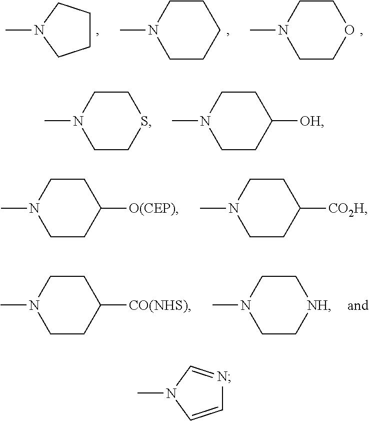

R¹² is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;

each Y is independently N, C—H, C—Z¹, or C—Z²;

each Z¹ is independently OCH₃, CN, or CF₃;

each Z² is independently CH₃, F, Cl, Br, or I;

m is an integer from 2 to 8; and

each n is independently an integer from 1 to 5.

In some embodiments, the present disclosure relates to the following compounds of formula (I):

(1) Compounds wherein A is
(a) CO₂R¹;
(b) CO²R²;
(c) C(O)NR³R⁴;
(d) CN;
(e) S(O)R⁵;
(f) SO₂R⁵;
(g) C(O)R⁶;
(h) C(O)R⁷; or
(i) CO(NHS);

(2) Compounds wherein B is
(a) B¹;
(b) B²;
(c) B³;
(d) B⁴; or
(e) B², B³, or B⁴;

(3) Compounds wherein each occurrence of X is
(a) N;
(b) C—H;
(c) C—Z¹; or
(d) C—Z²;

(4) Compounds wherein at least one occurrence of X is
(a) N;
(b) C—H;
(c) C—Z¹; or
(d) C—Z²;

(5) Compounds wherein each occurrence of Y is
(a) N;
(b) C—H;
(c) C—Z¹; or
(d) C—Z²; and

(6) Compounds wherein at least one occurrence of Y is
(a) N;
(b) C—H;
(c) C—Z¹; or
(d) C—Z².

In some embodiments, the present disclosure relates to compounds of formula (I) wherein, if each X is independently C—H or C—Z², and A is CO₂R¹, CN, or COR⁶, then B is B², B³, or B⁴

In some embodiments, the present disclosure relates to compounds of formula (I), wherein each X is C—H, A is CO₂R¹, and B is B², B³, or B⁴.

In some embodiments, the present disclosure relates to compounds of formula (I), wherein each X is C—H, A is CN, and B is B², B³, or B⁴.

In some embodiments, the present disclosure relates to compounds of formula (I), wherein each X is C—H, A is COR⁶, and B is B², B³, or B⁴.

In some embodiments, the present disclosure relates to compounds of formula (I), wherein at least one X is N or C—Z¹ and A is CO₂R¹, CN or COR⁶.

In some embodiments, the present disclosure relates to compounds of formula (I), wherein A is CO₂R², C(O)NR³R⁴, S(O)R⁵, SO₂R⁵, C(O)R⁷, or CO(NHS)

In some embodiments, the present disclosure relates to compounds of formula (I), wherein A is CO₂R¹ and R¹ is (C₁-C₁₂)alkyl.

In some embodiments, the present disclosure relates to compounds of formula (I), wherein A is CO₂R² and R² is CH₂CH₂(C₄-C₁₂)perfluoroalkyl or (CH₂CH₂O)ₙCH₂CH₂O(C₁-C₄)alkyl.

In some embodiments, the present disclosure relates to compounds of formula (I), wherein A is CO₂R² and R² is (CH₂)ₘOH, (CH₂)ₘSH, (CH₂)ₘNH₂, (CH₂)ₘCO(NHS) or —(CH₂)ₘ(N-maleimide).

In some embodiments, the present disclosure relates to compounds of formula (I), wherein A is CO₂R² and R² is —(CH₂CH₂O)ₙCH₂CH₂OH, —(CH₂CH₂O)ₙCH₂CH₂SH, —(CH₂CH₂O)ₙCH₂CH₂NH₂, —(CH₂CH₂O)ₙCH₂CH₂CO(NHS) or —(CH₂CH₂O)ₙCH₂CH₂(N-maleimide).

In some embodiments, the present disclosure relates to compounds of formula (I), wherein A is CO₂R² and R² is —(CH₂)ₘN₃, —(CH₂CH₂O)ₙCH₂CH₂N₃, —(CH₂)ₘOCH₂C≡CH or —(CH₂CH₂O)ₙCH₂CH₂OCH₂C≡CH.

In some embodiments, the present disclosure relates to compounds of formula (I), wherein A is CO₂R² and R² is —(CH₂)ₘO(CEP), —(CH₂CH₂O)ₙCH₂CH₂O(CEP), —(CH₂)₃OCH₂CH(OH)CH₂OH, —(CH₂CH₂O)ₙCH₂CH(OH)CH₂OH, —(CH₂)₃OCH₂CH(OH)CH₂O(DMT), —(CH₂CH₂O)ₙCH₂CH(OH)CH₂O(DMT), —(CH₂)₃OCH₂CH(O(CEP))CH₂O(DMT) or —(CH₂CH₂O)ₙCH₂CH(O(CEP))CH₂O(DMT).

In some embodiments, the present disclosure relates to compounds of formula (I), wherein A is C(O)NR³R⁴ and R³ is (C₁-C₁₂)alkyl.

In some embodiments, the present disclosure relates to compounds of formula (I), wherein A is C(O)NR³R⁴ and R³ is (CH₂)ₘOH, (CH₂)ₘSH, (CH₂)ₘNH₂, (CH₂)ₘCO(NHS) or —(CH₂)ₘ(N-maleimide).

In some embodiments, the present disclosure relates to compounds of formula (I), wherein A is C(O)NR³R⁴ and R³ is —(CH₂CH₂O)ₙCH₂CH₂OH, —(CH₂CH₂O)ₙCH₂CH₂SH, —(CH₂CH₂O)ₙCH₂CH₂NH₂, —(CH₂CH₂O)ₙCH₂CH₂CO(NHS) or —(CH₂CH₂O)ₙCH₂CH₂(N-maleimide).

In some embodiments, the present disclosure relates to compounds of formula (I), wherein A is C(O)NR³R⁴ and R³ is —(CH₂)ₘN₃, —(CH₂CH₂O)ₙCH₂CH₂N₃, —(CH₂)ₘOCH₂C≡CH or —(CH₂CH₂O)ₙCH₂CH₂OCH₂C≡CH.

In some embodiments, the present disclosure relates to compounds of formula (I), wherein A is C(O)NR³R⁴ and R³ is —(CH₂)ₘ0 (CEP), —(CH₂CH₂O)ₙCH₂CH₂O(CEP), —(CH₂)₃OCH₂CH(OH)CH₂OH, —(CH₂CH₂O)ₙCH₂CH(OH)CH₂OH, —(CH₂)₃OCH₂CH(OH)CH₂O(DMT), —(CH₂CH₂O)ₙCH₂CH(OH)CH₂O(DMT), —(CH₂)₃OCH₂CH(O(CEP))CH₂O(DMT) or —(CH₂CH₂O)ₙCH₂CH(O(CEP))CH₂O(DMT).

In some embodiments, the present disclosure relates to compounds of formula (I), wherein A is C(O)NR³R⁴ and R³ and R⁴ together with the nitrogen atom to which they are attached form a ring selected from the group consisting of

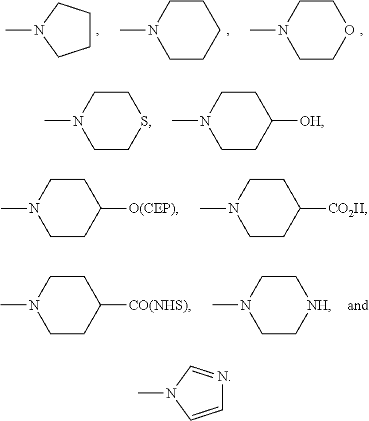

In some embodiments, the present disclosure relates to compounds of formula (I), wherein A is CO₂R¹, CO₂R², C(O)NR³R⁴, CN, S(O)R⁵, SO₂R⁵, C(O)R⁶, or C(O)R⁷; R² is CH₂CH₂(C₄-C₁₂)perfluoroalkyl; R³ is H, (C₁-C₁₂)alkyl, or CH₂CH₂(C₄-C₁₂)perfluoroalkyl; and R⁴ is H, (C₁-C₆)alkyl, CH₂CH₂(C₄-C₁₂)perfluoroalkyl, or (CH₂CH₂O)ₙCH₂CH₂O(C₁-C₄)alkyl, or R³ and R⁴ together with the nitrogen atom to which they are attached form a ring selected from the group consisting of

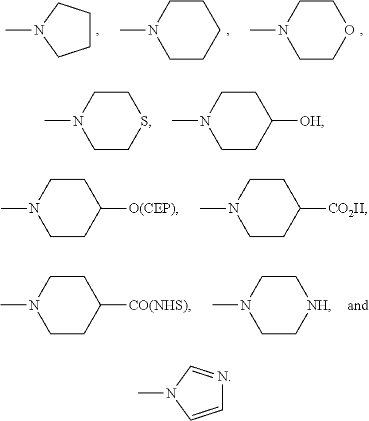

In some embodiments, the present disclosure relates to compounds of formula (I), wherein the compound of formula (I) is a compound of formula (Ia)

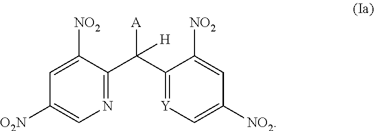

In some embodiments, the present disclosure relates to compounds of formula (I), wherein the compound of formula (I) is a compound of formula (Ib)

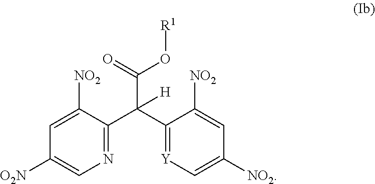

In some embodiments, the present disclosure relates to compounds of formula (I), wherein the compound of formula (I) is a compound of formula (Ic)

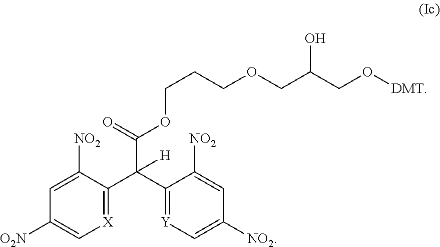

In some embodiments, the present disclosure relates to compounds of formula (I), wherein the compound of formula (I) is a compound of formula (Id)

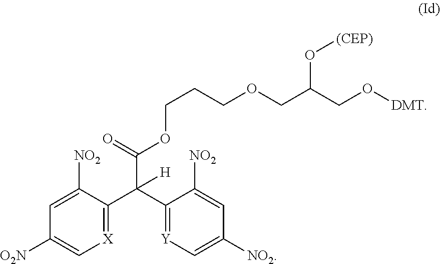

In some embodiments, the present disclosure relates to compounds of formula (I), wherein the compound of formula (I) is a compound of formula (Ie)

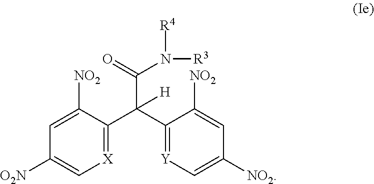

In some embodiments, the present disclosure relates to compounds of formula (I), wherein the compound of formula (I) is a compound of formula (If)

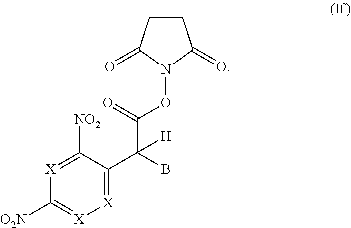

In some embodiments, the present disclosure relates to compounds of formula (I), wherein the compound of formula (I) is a compound of formula (Ig)

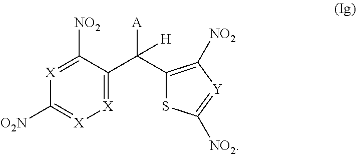

In some embodiments, the present disclosure relates to a compound of formula (I) is selected from the group consisting of:

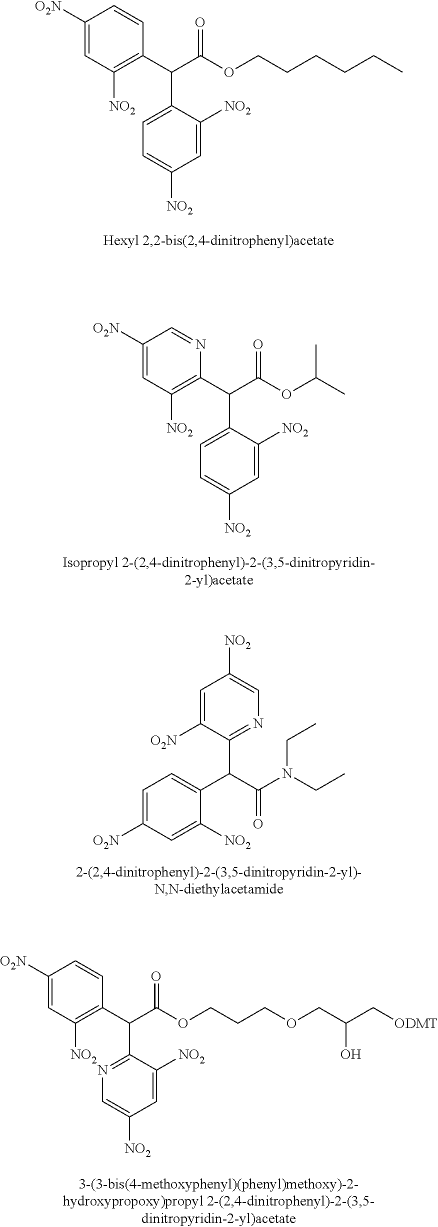

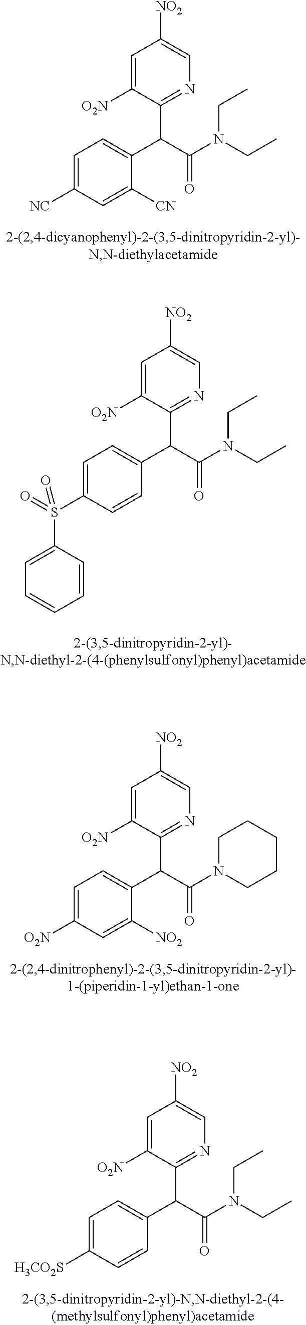

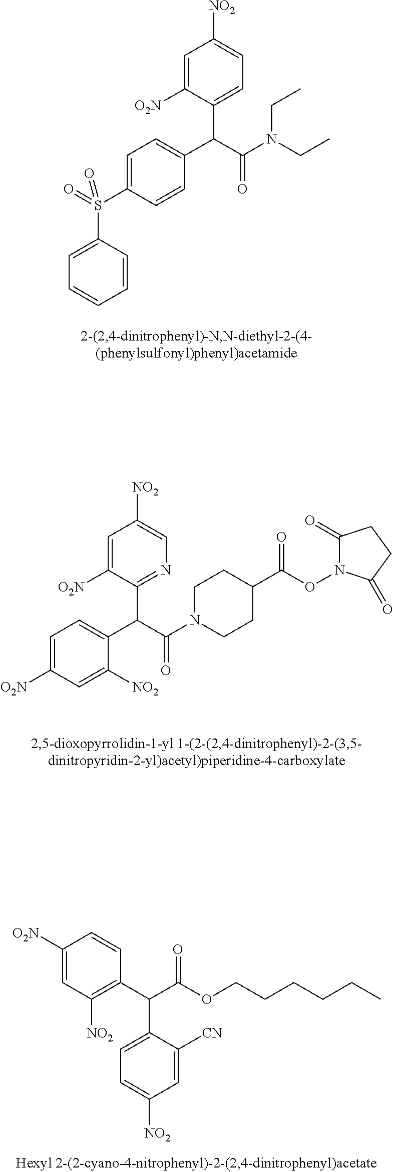 [SMILES: CCN(CC)C(=O)C(c1ccc(S(=O)(=O)c2ccccc2)cc1)c1ccc([N+](=O)[O-])cc1[N+](=O)[O-] ; O=C(ON1C(=O)CCC1=O)C1CCN(C(=O)C(c2ccc([N+](=O)[O-])cc2[N+](=O)[O-])c2ncc([N+](=O)[O-])cc2[N+](=O)[O-])CC1 ; CCCCCCOC(=O)C(c1ccc([N+](=O)[O-])cc1C#N)c1ccc([N+](=O)[O-])cc1[N+](=O)[O-]]

A combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week.

It will be apparent to one skilled in the art that certain compounds of this invention may exist in tautomeric forms, all such tautomeric forms of the compounds being within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools.

### Covalent Conjugates of Formula (II)

In another aspect, the present disclosure relates to a covalent conjugate of

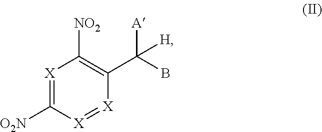

or a salt thereof, wherein,

A′ is CO₂R¹, CO₂R^{2′}, C(O)NR^{3′}R^{4′}, CN, S(O)R⁵, SO₂R⁵, C(O)R⁶, C(O)R⁷, CO(NHS), or CO-(L-[Conjugated Species]);

B is selected from the group consisting of B¹, B², B³, and B⁴; wherein

B¹ is

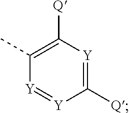

B² is selected from the group consisting of

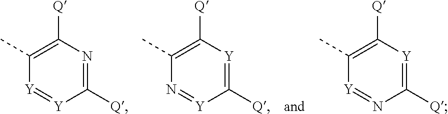

B³ is selected from the group consisting of

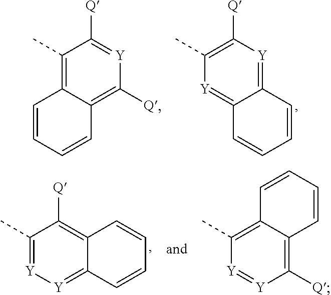

and

B⁴ is selected from the group consisting of

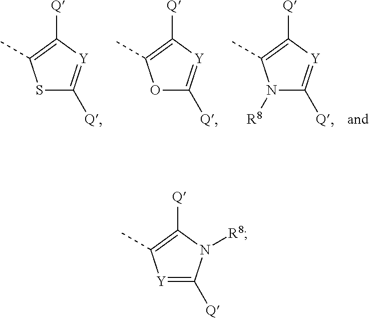

each X is independently N, C—H, C—Z¹, or C—Z²;

L is a single covalent bond or a covalent linkage having 1-50 non-hydrogen atoms selected from the group consisting of C, N, O, S and P and is composed of any combination of single, double, triple or aromatic bonds;

each Q′ is independently H, NO₂, Cl, Br, F, I, CH₃, OCH₃, CN, CO₂R⁹, C(O)NR^{10′}R^{11′}, SO₂R¹², SOR¹², CF₃, N₃, or CO-L-[Conjugated Species];

R¹ is H or (C₁-C₁₂)alkyl;

R^{2′} is CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂)ₘ-L-[Conjugated Species], (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂-L-[Conjugated Species], (CH₂CH₂O)ₙCH₂CH₂OCH₂C≡CH, (CH₂CH₂O)ₙCH₂-L-[Conjugated Species], (CH₂)₃OCH₂CH(OH)CH₂OH, (CH₂)₃OCH₂CH(OH)CH₂O(DMT), (CH₂)₃OCH₂CH(O(CEP))CH₂O(DMT), (CH₂)₃OCH₂CH(L-[Conjugated Species])CH₂OH, (CH₂)₃OCH₂CH(L-[Conjugated Species])CH₂-L-[Conjugated Species], (CH₂CH₂)ₙOCH₂CH(OH)CH₂OH, (CH₂CH₂)ₙOCH₂CH(OH)CH₂O(DMT), (CH₂CH₂)ₙOCH₂CH(O(CEP))CH₂O(DMT), (CH₂CH₂)ₙOCH₂CH(L-[Conjugated Species])CH₂OH, or (CH₂CH₂)ₙOCH₂CH(L-[Conjugated Species])CH₂-L-[Conjugated Species];

R^{3′} is H, (C₁-C₁₂)alkyl, CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂)ₘ-L-[Conjugated Species], (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂-L-[Conjugated Species], (CH₂CH₂O)ₙCH₂CH₂OCH₂C≡CH, (CH₂CH₂O)ₙCH₂-L-[Conjugated Species], (CH₂)₃OCH₂CH(OH)CH₂OH, (CH₂)₃OCH₂CH(OH)CH₂O(DMT), (CH₂)₃OCH₂CH(O(CEP))CH₂O(DMT), (CH₂)₃OCH₂CH(L-[Conjugated Species])CH₂OH, (CH₂)₃OCH₂CH(L-[Conjugated Species])CH₂-L-[Conjugated Species], (CH₂CH₂)ₙOCH₂CH(OH)CH₂OH, (CH₂CH₂)ₙOCH₂CH(OH)CH₂O(DMT), (CH₂CH₂)ₙOCH₂CH(O(CEP))CH₂O(DMT), (CH₂CH₂O)ₙOCH₂CH(L-[Conjugated Species])CH₂OH, or (CH₂CH₂O)ₙOCH₂CH(L-[Conjugated Species])CH₂-L-[Conjugated Species];

R^{4′} is H, (C₁-C₆)alkyl, CH₂CH₂(C₄-C₁₂)perfluoroalkyl, or (CH₂CH₂O)ₙCH₂CH₂O(C₁-C₄)alkyl, or R^{3′} and R^{4′} together with the nitrogen atom to which they are attached form a ring selected from the group consisting of

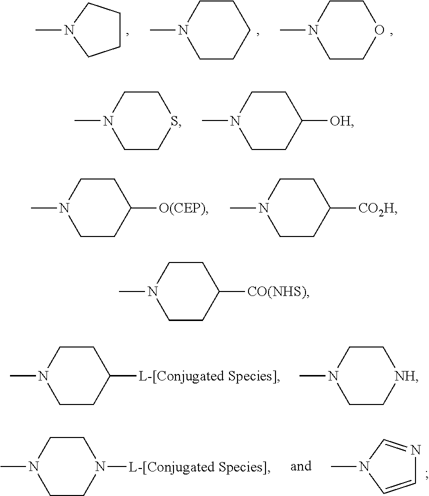

R⁵ is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;

R⁶ is H, (C₁-C₆)alkyl, or phenyl;

R⁷ is phenyl, wherein said phenyl is substituted with one to five substituents selected from the group consisting of CH₃, F, Cl, Br, I, OCH₃, OH, NO₂, CN, CF₃, N₃, and N(CH₃)₂;

R⁸ is H, (C₁-C₆)alkyl, CH₂CH₂(C₄-C₁₂)perfluoroalkyl), or (CH₂CH₂O)ₙCH₂CH₂O(C₁-C₄)alkyl;

R⁹ is H, (C₁-C₁₂)alkyl, CH₂CH₂(C₄-C₁₂)perfluoroalkyl), or (CH₂CH₂O)ₙCH₂CH₂O(C₁-C₄)alkyl;

R^{10′} is H, (C₁-C₁₂)alkyl, CH₂CH₂(C₄-C₁₂)perfluoroalkyl), or (CH₂CH₂O)ₙCH₂CH₂O(C₁-C₄)alkyl;

R^{11′} is H, (C₁-C₆)alkyl, or (CH₂CH₂O)ₙCH₂CH₂O(C₁-C₄)alkyl, or R^{10′} and R^{11′} together with the nitrogen atom to which they are attached form a ring selected from the group consisting of

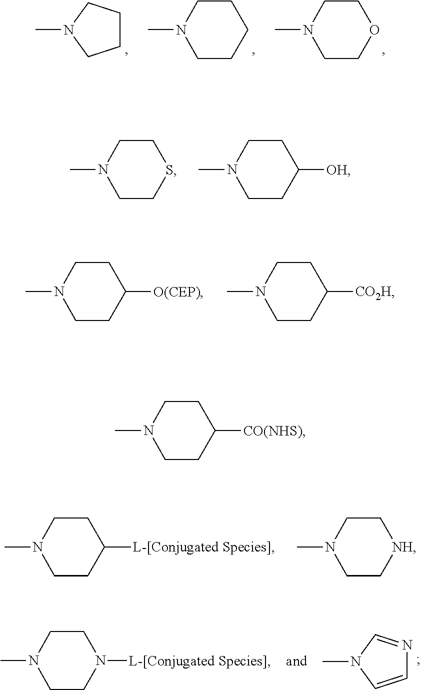

R¹² is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;

each Y is independently N, C—H, C—Z¹, or C—Z²;

each Z¹ is independently OCH₃, CN, or CF₃;

each Z² is independently CH₃, F, Cl, Br, or I;

m is an integer from 2 to 8; and

each n is independently an integer from 1 to 5;

wherein the covalent conjugate of formula (II) includes at least one occurrence of -L-[Conjugated Species].

In some embodiments, the present disclosure relates to a covalent conjugate of formula (II) wherein the variables A′, B, X, Q′, R¹, R^{2′}, R^{3′}, R^{4′}, R⁵, R⁶, R⁷, R⁸, R⁹, R^{10′}, R^{11′}, R¹², Y, Z¹, Z², m, and n are defined as A, B, X, Q, R¹, R², R³, R⁴, R⁵, R⁶, R⁷, R⁸, R⁹, R¹⁰, R¹¹, R¹², Y, Z¹, Z², m, and n are defined above with respect to the compounds of formula (I).

In some embodiments, L is a single covalent bond or a covalent linkage having 1-30 non-hydrogen atoms selected from the group consisting of C, N, O, S and P and is composed of any combination of single, double, triple or aromatic bonds.

In some embodiments, the present disclosure relates to a covalent conjugate of formula (II), wherein the Conjugated Species is a protein, an antibody, or a polypeptide.

In some embodiments, the present disclosure relates to a covalent conjugate of formula (II), wherein the Conjugated Species is a nucleic acid or an oligonucleotide.

In some embodiments, the present disclosure relates to a covalent conjugate of formula (II), wherein the Conjugated Species is a solid support.

In some embodiments, the present disclosure relates to a covalent conjugate of formula (II), wherein the Conjugated Species is a soluble polymer or an insoluble polymer.

In some embodiments, the present disclosure relates to a covalent conjugate of formula (II), wherein the Conjugated Species is a dendrimer.

In some embodiments, the present disclosure relates to a covalent conjugate of formula (II), wherein the Conjugated Species is a polysaccharide.

In some embodiments, the present disclosure relates to a covalent conjugate of formula (II), wherein the Conjugated Species is a fatty acid, a lipid or a phospholipid.

The covalent conjugates of formula (II) may be derived from compounds of formula (I) containing functional groups that facilitate covalent attachment to a Conjugated Species. For example, such functional groups are present in the compounds of formula (I), wherein:

A is CO(NHS) or CO₂R¹ and R¹ is H;

A is CO₂R² and R² is (CH₂)ₘOH, (CH₂)ₘSH, (CH₂)ₘN₃, (CH₂)ₘOCH₂C≡CH, (CH₂)ₘNH₂, (CH₂)ₘCO(NHS), (CH₂)ₘ(N-maleimide), (CH₂)ₘO(CEP), (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂OCH₂C≡CH, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);

A is CONR³R⁴ and R³ is (CH₂)ₘOH, (CH₂)ₘSH, (CH₂)ₘN₃, (CH₂)ₘOCH₂C≡CH, (CH₂)ₘNH₂, (CH₂)ₘCO(NHS), (CH₂)ₘ(N-maleimide), (CH₂)ₘO(CEP), (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂OCH₂C≡CH, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) (CH₂CH₂)ₙOCH₂CH(O(CEP))CH₂O(DMT) or R³ and R⁴ together with the nitrogen atom to which they are attached form a ring that is

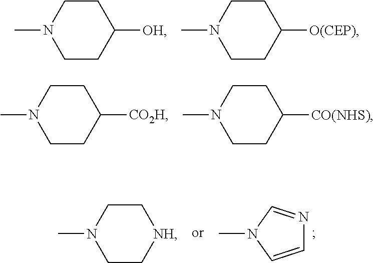

or

Q is CONR¹⁰R¹¹, and R¹⁰ and R¹¹ together with the nitrogen atom to which they are attached form a ring that is

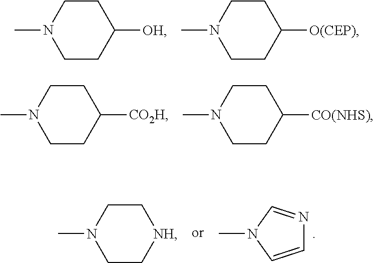

Covalent attachment of compounds of formula (I) to Conjugated Species to form covalent conjugates of formula (II) may be accomplished using procedures that are well known in the art. For example, covalent attachment may be achieved by formation of one or more covalent bonds by common chemical reactions that create one or more instances of ether, thioether, disulfide, amide, carbamate, urea, carboxylic acid ester, carboxylic acid thioester, 1,2,3-triazole, phosphate ester, or phosphate diester groups within L, at the junction between the compound of formula (I) and the Conjugated Species. The chemical reactions that are used to construct conjugates are well known to those skilled in the art. For example, U.S. Pat. No. 8,546,590, the entire contents of which are incorporated by reference, describes conjugates of polar fluorescent dyes with oligonucleotides. As additional examples, U.S. Pat. Nos. 7,968,586 and 8,586,049, the entire contents of which are incorporated by reference, describe drug-antibody conjugates.

### Use of Compounds of Formula (I) and Covalent Conjugates of Formula (II) as Fluorescence Quenchers

In a further aspect, the present disclosure relates to a method for detecting a change in the pH of a mixture. The method comprises (i) providing a mixture comprising a fluorescence donor and either a compound of formula (I) or a salt thereof or a covalent conjugate of formula (II) or a salt thereof; (ii) irradiating the resulting mixture at a first time with light having a wavelength suitable to excite the fluorescence donor; (iii) measuring detectable fluorescence emitted by the fluorescence donor while irradiating the mixture at said first time; (iv) irradiating the resulting mixture at a second time with light having a wavelength suitable to excite the fluorescence donor; (v) measuring detectable fluorescence emitted by the fluorescence donor while irradiating the mixture at said second time; (vi) comparing the detectable fluorescence emitted by the fluorescence donor at said first time with the detectable fluorescence emitted by the fluorescence donor at said second time to detect a change in the pH of the mixture; wherein a decrease in detectable fluorescence at said second time relative to said first time indicates an increase in the pH of the mixture, and wherein an increase in detectable fluorescence at said second time relative to said first time indicates a decrease in the pH of the mixture.

In some embodiments, the compound of formula (I), or salt thereof, or the covalent conjugate of formula (II), or salt thereof, is in a non-quenching state at the first time and is in a quenching state at the second time.

In some embodiments, the compound of formula (I), or salt thereof, or the covalent conjugate of formula (II), or salt thereof, is in a quenching state at the first time and is in a non-quenching state at the second time.

In some embodiments, the method further comprises continuously irradiating the mixture between the first time and the second time.

In some embodiments, the method further comprises continuously measuring detectable fluorescence emitted by the fluorescence donor while irradiating the mixture between the first time and the second time.

In some embodiments, the method further comprises irradiating the resulting mixture at a third time with light having a wavelength suitable to excite the fluorescence donor; measuring detectable fluorescence emitted by the fluorescence donor while irradiating the mixture at said third time; and comparing the detectable fluorescence emitted by the fluorescence donor at the third time with the detectable fluorescence emitted by the fluorescence donor at the first and second times to detect a further change in the pH of the mixture.

In some embodiments, the method further comprises continuously irradiating the mixture between the second time and the third time.

In some embodiments, the method further comprises continuously measuring detectable fluorescence emitted by the fluorescence donor while irradiating the mixture between the second time and the third time.

Fluorescence donors suitable for use in the disclosed method include without limitation fluorescein, tetrachlororfluorescein (TET), hexachlorofluorescein (HEX), dichlorodimethoxyfluorescein (Joe), tetramethylrhodamine, Texas Red, Rhodamine-X (ROX), Cy3, Cy5, Cy5.5, and the like. Preferably, the fluorescence donor has an emission wavelength between about 500 and about 750 nm.

The disclosed method may be conducted in any solvent or solvent mixture. Suitable solvents include but are not limited to water, water-miscible organic solvents (e.g., DMSO, DMF, DMA, MeOH, EtOH MeCN, THF), and water-immiscible organic solvents (e.g., diethyl ether, mineral oil, petroleum ether, methylene chloride), and the like. In the practice of the method described herein, it will be understood that the fluorescence donors and the compounds of formula (I) or covalent conjugates of formula (II) need not be dissolved in the solvent. For example, the fluorescence donors and the compounds of formula (I) or covalent conjugates of formula (II) may be suspended or in emulsion in the solvent.

Any suitable source of electromagnetic radiation may be used in the irradiating steps of the disclosed method. Suitable radiation sources are well known to persons skilled in the art of fluorescence assays. The wavelength of radiation used in a particular embodiment of the method depends on the absorption spectrum of the fluorescence donor, as is well-known in the art. In some embodiments, the wavelength of radiation is between about 350 nm and about 1000 nm.

Suitable instruments for measuring detectable fluorescence in accordance with the disclosed method are well-known to persons skilled in the art of fluorescence assays.

Without wishing to be bound by any theory, it is believed, as shown in Scheme 1, that the deprotonation of a compound of formula (I) results in the formation of an intensely colored anion (I-A) that acts as a fluorescence quencher. Conversely, the protonation of the anion (I) is believed to afford a colorless compound of formula (I), which does not act as a fluorescence quencher. The covalent conjugates of formula (II) are believed to behave in a similar way.

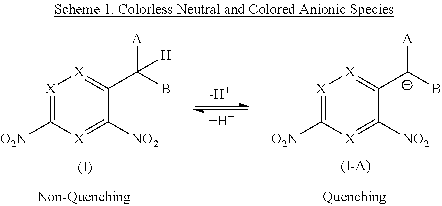

The fluorescence quenching properties of the compounds of formula (I), and their corresponding anions (I-A), depend on the chemical structures of the compounds. For example, the pKa of each compound of formula (I), and thus the pH at which each compound becomes colored, depends upon the nature of groups A, B, and X. Generally, compounds having electron-withdrawing A, B, and X groups have lower pKa values and become colored at lower pH. The light absorption spectra of the anions of formula (I-A) also depend on structure. Table 1 reports the pKa values of three compounds of formula (I). Table 1 also reports the maximal absorption wavelength (A max) and molar extinction coefficient (E) of the anions derived from the listed compounds.

TABLE 1

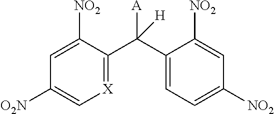

| Compound | A | X | pKa | λ max (nM) | ε cm⁻¹ M⁻¹ |
|---|---|---|---|---|---|
| 1 | CO₂(n-C₆H₁₃) | C—H | 7.8 | 652 | — |
| 2 | CO₂(i-C₃H₇) | N | 4.3 | 592 | 29,950 |
| 3 | CON(Et)₂ | N | 5.5 | 616 | 22,000 |

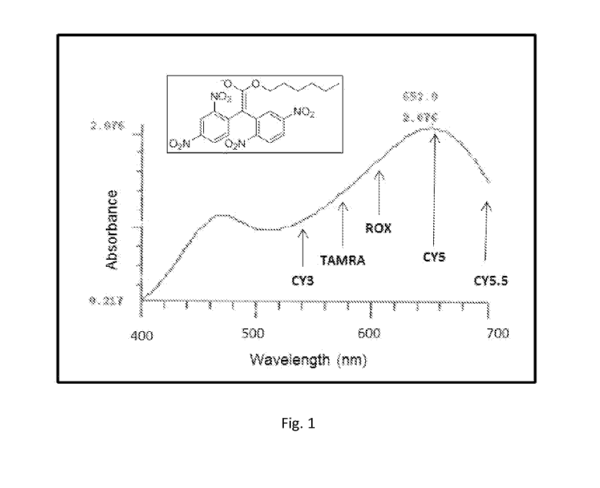

The compounds of formula (I) and covalent conjugates of formula (II) are useful as components in fluorescence based signal transduction systems because they possess little or no background fluorescence and can modulate a fluorescence signal in response to changes in pH. The anions derived from these compounds also have strong extinction coefficients and broad light absorption spectra, such that they are able to quench the fluorescence produced by a variety of fluorophores. For example, FIG. 1 depicts the absorption spectrum of the anion derived from compound 1 in relation to the light emission maxima of several commercially available fluorophores.

The compounds of formula (I) and covalent conjugates of formula (II) differ from many available quencher dyes, such as Dabcyl, Dabsyl, BHQ-0®, BHQ-1®, BHQ-2®, BHQ-3®, BBQ-650®, Iowa Black® FQ, Iowa Black® RQ, IQ2™, and IQ4™ in at least three ways. First, the compounds of formula (I) and covalent conjugates of formula (II) can be turned-on (become colored) to act as quenchers of fluorescence or turned-off (become colorless) reversibly by adjusting the pH of the environment. By contrast, the aforementioned commercially available dyes always display the intense color that provides the fluorescence quenching effect. In order to turn-off their quenching effect, they must be removed from the proximity of the fluorophore. Second, compounds of formula (I) and covalent conjugates of formula (II) have very different chemical structures from the commercially available dyes. For example, the compounds of formula (I) and covalent conjugates of formula (II) lack the diazo-arene (¹Ar—N═N—Ar²) functional group present in all of the commercially available dyes. Third, many of the commercially available quencher dyes are sensitive to thiols, which cause gradual degradation of their quenching effect. The compounds of formula (I) and covalent conjugates of formula (II) do not display this thiol sensitivity. This stability is important because dithiothreitol, 2-mercaptoethanol, glutathione and other thiols are often employed as biological molecule stabilizers.

### Synthesis of the Compounds of Formula (I)

General methods for preparing compounds of formula (I) are outlined in Schemes 2-6. The starting materials employed in the syntheses outlined below can be obtained using straightforward synthetic transformations well known to the person of ordinary skill in the art. Compounds of formula (I) not accessible via the methods set forth in Schemes 2-6 are accessible using other common chemical transformations.

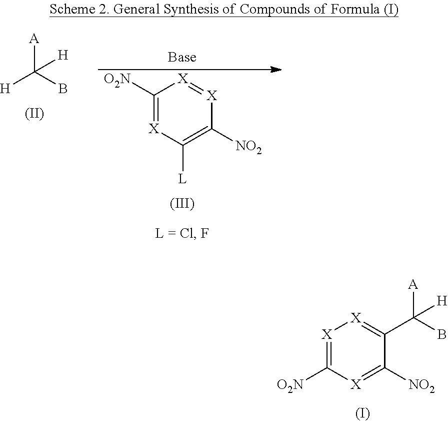

As shown in Scheme 2, treatment of a compound of formula (II) with a chloro- or fluoro-dinitroarene of formula (III) affords the compound of formula (I).

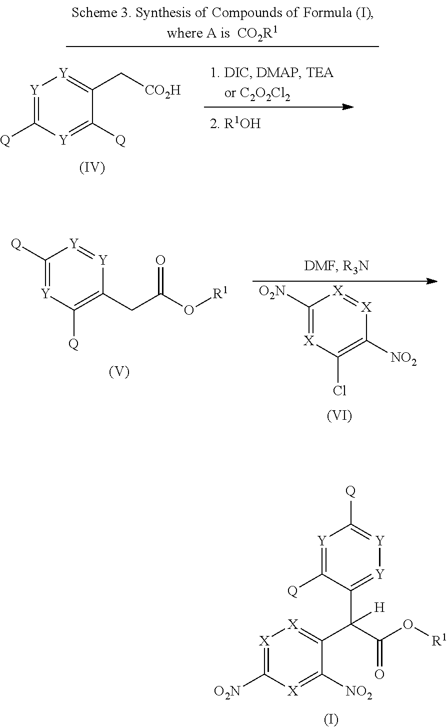

Scheme 3 depicts a synthesis of compounds of formula (I) where A is CO₂R¹. A carboxylic acid of formula (IV) is treated with DIC, DMAP, and a base (e.g., TEA) or oxalyl chloride and, optionally, a base (e.g., TEA), followed by treatment with an alcohol (R¹OH), to afford an ester of formula (V). The ester of formula (V), in turn, reacts with a chlorodinitroarene of formula (VI) in the presence of a base (e.g., a trialkylamine, R₃N) to afford the compound of formula (I).

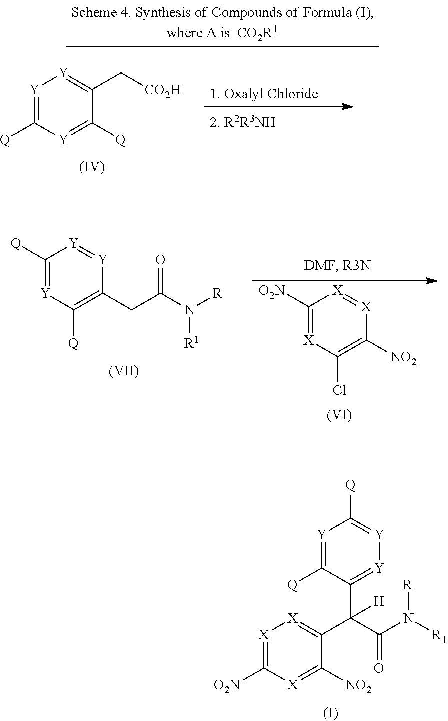

Scheme 4 depicts a synthesis of compounds of formula (I) where A is CONR²R³. A carboxylic acid of formula (IV) is treated with oxalyl chloride, followed by treatment with an amine (R²R³NH) and, optionally, a base (e.g., TEA), to afford an amide of formula (VII). The amide of formula (VII), in turn, reacts with a chlorodinitroarene of formula (VI) in the presence of a base (e.g., a trialkylamine, R₃N) to afford the compound of formula (I).

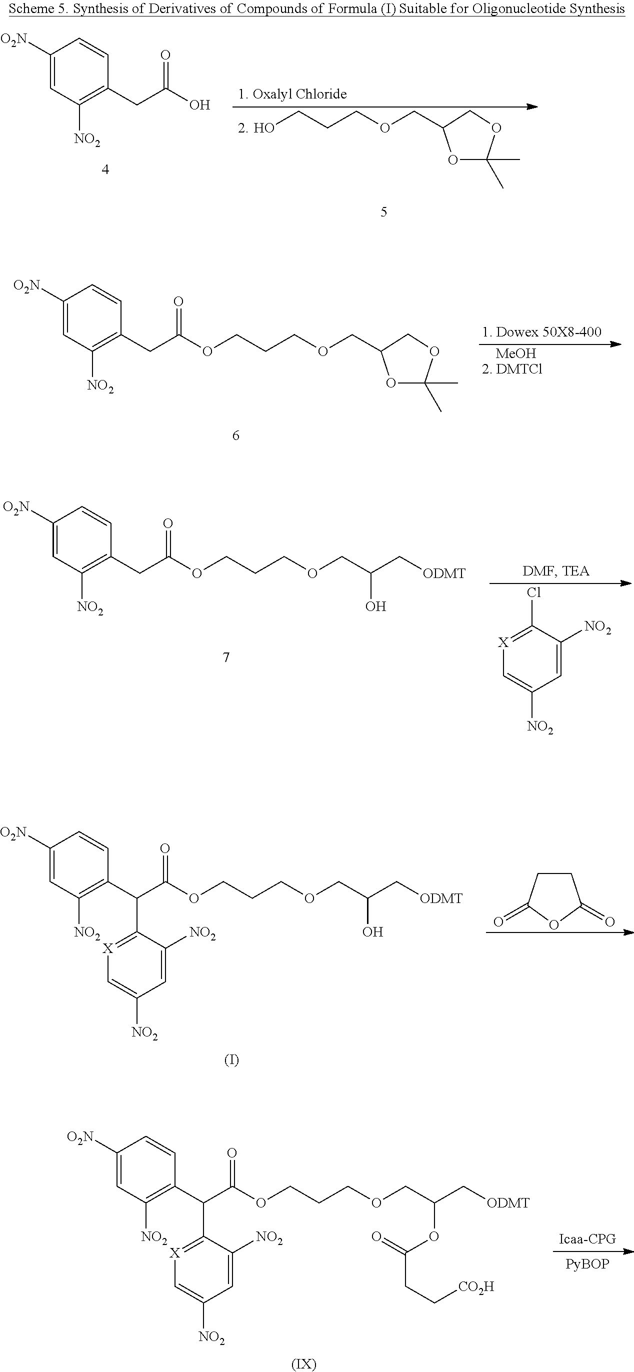

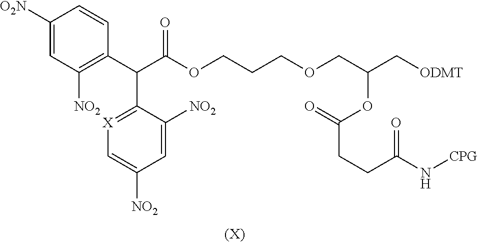

Scheme 5 illustrates the synthesis of a compound of formula (I) that can be elaborated into a compound of formula (X). The compound of formula (X) is connected to a solid support and is suitable for use in oligonucleotide synthesis. The methods outlined in Scheme 5 are generally applicable for the synthesis of compounds of formula (I) in which A is CO₂R¹ and R¹ is (CH₂)₃OCH₂CH(OH)CH₂O(DMT) or (CH₂CH₂)ₙOCH₂CH(OH)CH₂O(DMT). Dinitrophenylacetic acid 4 is treated with oxalyl chloride, followed by alcohol 5, optionally in the presence of a base, to afford ester 6. Ester 6 is treated with Dowex 50X8-400, followed by DMT-Cl, optionally in the presence of a base, to afford mono-DMT protected diol 7. Compound 7 is treated with a chlorodinitroarene of formula (VIII) in the presence of a base (e.g., TEA) to afford the compound of formula (I). The compound of formula (I) is treated with succinic anhydride and, optionally, pyridine to afford a succinic acid monoester intermediate of formula (IX). The succinic acid monoester intermediate of formula (IX) is treated with one of a variety of amide bond coupling reagents, such as PYBOP (benzotriazol-1-yl-oxy-tris-pyrrolidino-phosphonium hexafluorophosphate) and diisopropylethylamine, followed by addition of aminopropyl-CPG to afford the CPG-supported product of formula (X). Lcaa-CPG also may be used in place of aminopropyl-CPG to afford the corresponding Icaa-CPG product.

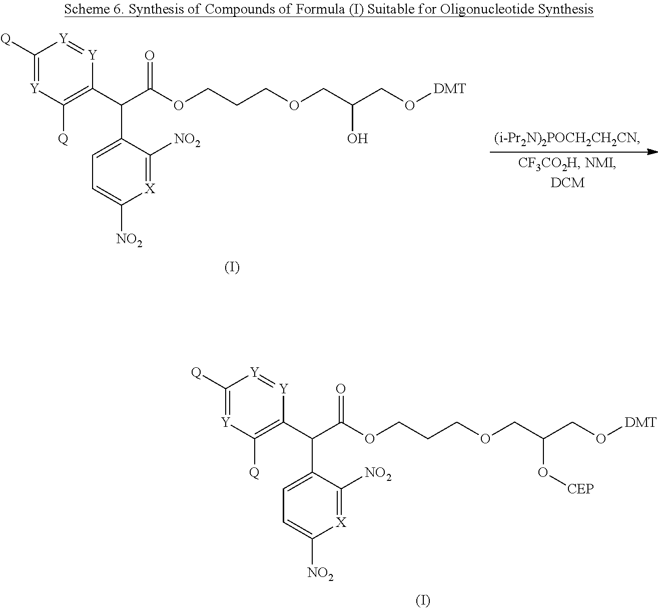

Scheme 6 outlines a method for incorporating a CEP group into a compound of formula (I) to provide a compound suitable for oligonucleotide synthesis. A compound of formula (I) having a free hydroxyl group is treated with 2-cyanoethyl-bis(diisopropylamino)phosphite in the presence of trifluoroacetic acid and a base, such as N-methylimidazole, to afford a compound of formula (I) in which the free hydroxyl group has been protected with a CEP group. The method outlined in Scheme 6 is generally applicable for synthesis of compounds of formula (I) in which A is CO₂R¹ and R¹ is (CH₂)₃OCH₂CH(O(CEP))CH₂O(DMT) or (CH₂CH₂)ₙOCH₂CH(O(CEP))CH₂O(DMT).

### EXAMPLES

In order that this disclosure may be more fully understood, the following examples are set forth. These examples are for the purpose of illustration only and are not to be construed as limiting the scope of the disclosure in any way.

### Example 1

### Hexyl 2,2-bis(2,4-dinitrophenyl)acetate (1)

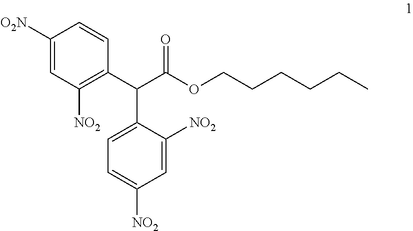 [SMILES: CCCCCCOC(=O)C(c1ccc([N+](=O)[O-])cc1[N+](=O)[O-])c1ccc([N+](=O)[O-])cc1[N+](=O)[O-]]

### Step 1. Synthesis of Hexyl 2-(2,4-dinitrophenyl)acetate

A suspension of 2-(2,4-dinitrophenyl)acetic acid (2.26 g, 10.0 mmol), 1-hexanol (2.5 mL, 20.0 mmol), and DMAP (976 mg, 8.0 mmol) in dichloromethane (30 mL) was cooled in an ice-water bath, under a nitrogen atmosphere, and treated with diisopropylcarbodiimide (1.72 mL, 11 mmol). The reaction mix became red-orange in color. The reaction mix was allowed to warm to room temperature and stirred over the weekend. The reaction mixture was diluted with saturated NH₄Cl, the organic layer was separated, dried (Na₂SO₄), and concentrated to give a brown oil. The crude product was purified by flash chromatography (50 g SiO₂), eluting with dichloromethane, to give 1.0 g (32%) of hexyl 2-(2,4-dinitrophenyl)acetate: R_{f}=0.65 (dichloromethane).

### Step 2. Synthesis of Hexyl 2,2-bis(2,4-dinitrophenyl)acetate

A solution of hexyl 2-(2,4-dinitrophenyl)acetate (310 mg, 1.0 mmol) and 2-chloro-1,4-dinitrobenzene (202 mg, 1.0 mmol) in dry DMF (10 mL) was treated with triethylamine (280 uL, 2.0 mmol). The dark green solution was allowed to stir at rt overnight. The reaction mixture was diluted with water (30-40 mL) and treated with 1M hydrochloric acid until pH=1.0. The mixture was extracted with dichloromethane. The organic extracts were washed with saturated NH₄Cl solution, dried (Na₂SO₄), and concentrated to give a brown oil. The crude product was purified by MPLC (50 g SiO₂), eluting with dichloromethane to give 300 mg (63%) of a slightly yellow powder. The product was precipitated from DCM/MeOH to give hexyl 2,2-bis(2,4-dinitrophenyl)acetate as a colorless powder: MS ES-API m/z=475.5 [M−H]⁻.

### Example 2

### Isopropyl 2-(2,4-dinitrophenyl)-2-(3,5-dinitropyridin-2-yl)acetate (2)

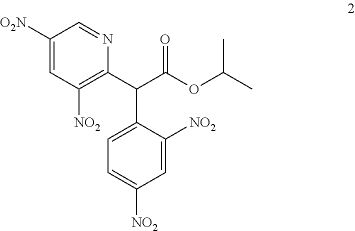 [SMILES: CC(C)OC(=O)C(c1ccc([N+](=O)[O-])cc1[N+](=O)[O-])c1ncc([N+](=O)[O-])cc1[N+](=O)[O-]]

### Step 1. Synthesis of Isopropyl 2-(2,4-dinitrophenyl)acetate

A suspension of 2-(2,4-dinitrophenyl)acetic acid (5.0 g, 22 mmol) in dichloromethane (50 mL) was treated with neat oxalyl chloride (2.83 mL, 33 mmol) and dry DMF (2 drops). The evolution of gas was noted. The reaction mix was allowed to stir at room temperature for 4 hours. An additional portion of oxalyl chloride (1 mL, 11 mmol) was added to the reaction mixture and stirring was continued for 1 hour. The reaction mixture became homogeneous. The solution was concentrated to an orange oil then concentrated from dry toluene (15 mL) to give crude 2-(2,4-dinitrophenyl)acetyl chloride as a yellow-orange solid. A solution of isopropanol (1 mL, 13 mmol) in DCM (5 mL) was added to a solution of the crude 2-(2,4-dinitrophenyl)acetyl chloride (250 mg, 1.0 mmol) in DCM (2 mL). The reaction mixture was concentrated to give a dark solid. The crude solid was partitioned between DCM and 1M HCl. The organic layer was separated, dried (Na₂SO₄), and concentrated to an orange oil. Purification by flash chromatography (4 g SiO₂) eluting with hexanes/dichloromethane/acetone (50:50:0.5) to give 99 mg (36%) of isopropyl 2-(2,4-dinitrophenyl)acetate: LC-MS ES-API m/z=267.2 [M−H]⁻

### Step 2. Synthesis Isopropyl 2-(2,4-dinitrophenyl)-2-(3,5-dinitropyridin-2-yl)acetate

A mixture of Isopropyl 2-(2,4-dinitrophenyl)acetate (40 mg, 0.15 mmol) and 2-chloro-3,5-dinitropyridine (30 mg, 0.15 mmol) in dry DMF (1.5) was treated with TEA (500 uL, 3.6 mmol). The resulting dark mixture was allowed to stir at room temperature overnight. The dark violet solution was diluted with ethyl acetate and pH 4 buffer. The mixture was treated with 1M HCl until the dark color dissipated (pH 4). The organic layer was separated, washed with saturated NaCl solution, dried (Na₂SO₄), and concentrated to give an orange oil. Purification by flash chromatography (4 g SiO₂), eluting with hexanes/dichloromethane/acetone (50:50:1) to give 28 mg (42%) of isopropyl 2-(2,4-dinitrophenyl)-2-(3,5-dinitropyridin-2-yl)acetate: LC-MS ES-API m/z=434.0 [M−H]⁻.

### Example 3

### 2-(2,4-dinitrophenyl)-2-(3,5-dinitropyridin-2-yl)-N,N-diethylacetamide (3)

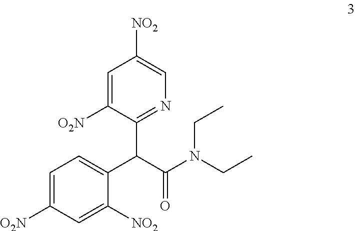 [SMILES: CCN(CC)C(=O)C(c1ccc([N+](=O)[O-])cc1[N+](=O)[O-])c1ncc([N+](=O)[O-])cc1[N+](=O)[O-]]

### Step 1. Synthesis of 2-(2,4-dinitrophenyl)-N,N-diethylacetamide

A suspension of 2-(2,4-dinitrophenyl)acetic acid (5.0 g, 22 mmol) in dichloromethane (50 mL) was treated with neat oxalyl chloride (2.83 mL, 33 mmol) and dry DMF (2 drops). The evolution of gas was noted. The reaction mix was allowed to stir at room temperature for 4 h. An additional portion of oxalyl chloride (1 mL, 11 mmol) was added to the reaction mixture, and stirring was continued for 1 h. The reaction mixture became homogeneous. The solution was concentrated to an orange oil then concentrated from dry toluene (15 mL) to give crude 2-(2,4-dinitrophenyl)acetyl chloride as a yellow-orange solid. A solution of diethylamine (0.5 mL, 4.8 mmol) in DCM (5 mL) was added to a solution of the crude 2-(2,4-dinitrophenyl)acetyl chloride (250 mg, 1.0 mmol) in DCM (2 mL). The reaction mixture was concentrated to give a dark solid. The solid was partitioned between DCM and 1M HCl. The organic layer was separated, dried (Na₂SO₄), and concentrated to give the crude product. Purification by flash chromatography (5 g SiO₂), eluting with dichloromethane/acetone (99:1) gave 100 mg (33%) of 2-(2,4-dinitrophenyl)-N,N-diethylacetamide: LC-MS ES-API m/z=282 [M−H]⁻

### Step 2. Synthesis of 2-(2,4-dinitrophenyl)-2-(3,5-dinitropyridin-2-yl)-N,N-diethylacetamide

A mixture of 2-(2,4-dinitrophenyl)-N,N-diethylacetamide (28 mg, 0.10 mmol) and 2-chloro-3,5-dinitropyridine (20.2 mg, 0.10 mmol) in dry DMF (1.0 mL) was treated with triethylamine (500 uL, 3.6 mmol). The resulting dark red mixture was allowed to stir at room temperature for 2 h. The dark violet solution was diluted with ethyl acetate and pH 4 buffer. The mixture was treated with 1M HCl until the dark color dissipated (pH 3). The organic layer was separated, dried (Na₂SO₄), and concentrated. Purification by flash chromatography (4 g SiO₂), eluting with hexanes/dichloromethane/acetone (50:50:2) to give 35 mg (78%) of 2-(2,4-dinitrophenyl)-2-(3,5-dinitropyridin-2-yl)-N,N-diethylacetamide as a colorless foam: LC-MS ES-API m/z=449 [M+H]+.

### Example 4

### 4-((1-(bis(4-methoxyphenyl)(phenyl)methoxy)-3-(3-(2-(2,4-dinitrophenyl)-2-(3,5-dinitropyridin-2-yl)acetoxy)propoxy)propan-2-yl)oxy)-4-oxobutanoic acid

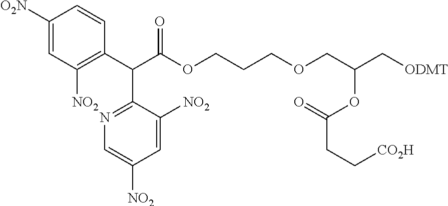

### Step 1. Synthesis of 3-((2,2-dimethyl-1,3-dioxolan-4-yl)methoxy)propyl 2-(2,4-dinitrophenyl)acetate

A suspension of 2-(2,4-dinitrophenyl)acetic acid (904 mg, 4 mmol) in dichloromethane (30 mL) was cooled in an ice-water bath and treated with neat oxalyl chloride (514 uL, 6 mmol) and dry DMF (1 drop). The cooling bath was removed and the reaction mix was allowed to stir at room temperature for 4 h. The solution was concentrated to a red oil then concentrated from dry DCE (5 mL) to give crude 2-(2,4-dinitrophenyl)acetyl chloride. A solution of the crude acid chloride in DCE (3 mL) was added dropwise to a room temperature solution of 3-((2,2-dimethyl-1,3-dioxolan-4-yl)methoxy)propan-1-ol (761 mg, 4 mmol) and TEA (2.0 mL mL, 14 mmol) in DCE (5 mL). The purple reaction mixture was diluted with DCM and washed with a mixture of saturated NH₄Cl-1M HCl (pH<2). The organic layer was removed, washed with ½-saturated brine, dried (Na₂SO₄), and concentrated to give the crude product. Purification by flash chromatography (100 g silica gel) eluting with EtOAc/dichloromethane (5-10%) gave 738 mg (46%) of 3-((2,2-dimethyl-1,3-dioxolan-4-yl)methoxy)propyl 2-(2,4-dinitrophenyl)acetate: LC-MS ES-API m/z=397 [M−H]⁻

### Step 2. Synthesis of 3-(3-(bis(4-methoxyphenyl)(phenyl)methoxy)-2-hydroxypropoxy)propyl 2-(2,4-dinitrophenyl)acetate

A solution of 3-((2,2-dimethyl-1,3-dioxolan-4-yl)methoxy)propyl 2-(2,4-dinitrophenyl)acetate (1.6 g, 4.0 mmol) in MeOH/water (20 mL, 9:1) was treated with Dowex 50wx8 resin (acid form). The suspension was mixed by rotating the flask in a 40° C. water bath (4 h). The reaction mixture was filtered to remove the resin. The filtrate was concentrated to an oil then co-evaporated from pyridine (2×). A solution of the crude diol in DCM/pyridine (36 mL, 2:1) was cooled in an ice-water bath under a nitrogen atmosphere and treated with a solution of DMT-CI in DCM over 30 min. The cooling bath was removed and stirring was continued for 4 h. The reaction mixture was quenched with MeOH (1 mL) then concentrated to a crude oil. Purification by flash chromatography (100 g silica gel) eluting with acetone/dichloromethane (4%) gave 1.46 g (55%) of 3-(3-(bis(4-methoxyphenyl)(phenyl)methoxy)-2-hydroxypropoxy)propyl 2-(2,4-dinitrophenyl)acetate: LC-MS ES-API m/z=659.2 [M−H]⁻

### Step 3. Synthesis of 3-(3-(bis(4-methoxyphenyl)(phenyl)methoxy)-2-hydroxypropoxy)propyl 2-(2,4-dinitrophenyl)-2-(3,5-dinitropyridin-2-yl)acetate

A solution of 3-(3-(bis(4-methoxyphenyl)(phenyl)methoxy)-2-hydroxypropoxy)propyl 2-(2,4-dinitrophenyl)acetate (300 mg, 0.45 mmol) in dry DMF (5 mL) was treated with 2-chloro-3,5-dinitropyridine (100 mg, 0.49 mmol) and triethylamine (5 mL). The mixture turns black then slowly becomes violet in color. The reaction mixture was poured into a pH 4 buffer and treated with 0.1M citric acid until the violet color dissipated. The mixture was extracted with ethyl acetate. The organic layer was separated, washed with a pH 6 buffer then washed with and saturated brine, dried (Na₂SO₄), and concentrated to a crude oil. Purification by flash chromatography (12 g silica gel) eluting with acetone/dichloromethane (3:97) gave 230 mg (55%) of 3-(3-(bis(4-methoxyphenyl)(phenyl)methoxy)-2-hydroxypropoxy)propyl 2-(2,4-dinitrophenyl)-2-(3,5-dinitropyridin-2-yl)acetate as a yellow foam: LC-MS ES-API m/z=826.4 [M−H]⁻

### Step 4. Synthesis of 4-((1-(bis(4-methoxyphenyl)(phenyl)methoxy)-3-(3-(2-(2,4-dinitrophenyl)-2-(3,5-dinitropyridin-2-yl)acetoxy)propoxy)propan-2-yl)oxy)-4-oxobutanoic acid

3-(3-(bis(4-methoxyphenyl)(phenyl)methoxy)-2-hydroxypropoxy)propyl 2-(2,4-dinitrophenyl)-2-(3,5-dinitropyridin-2-yl)acetate (230 mg, 0.28 mmol), DMAP (34 mg, 0.28 mmol), and succinic anhydride (83 mg, 0.83 mmol) were combined in dry pyridine (5 mL). The resulting dark blue solution was stirred at 50° C. for 2 h then at 55° C. overnight. The reaction mixture was cooled in an ice-water bath and quenched with water (0.2 mL). The reaction mixture was concentrated to a dark oil, dissolved in DCM (50 mL), washed with water then brine, dried (Na₂SO₄), and concentrated to a crude oil. Purification by flash chromatography (8 g silica gel) eluting with MeOH/dichloromethane (1:99 to 1:20) gave 37 mg (14%) of 4-((1-(bis(4-methoxyphenyl)(phenyl)methoxy)-3-(3-(2-(2,4-dinitrophenyl)-2-(3,5-dinitropyridin-2-yl)acetoxy)propoxy)propan-2-yl)oxy)-4-oxobutanoic acid as a tan solid: LC-MS ES-API m/z=926.4 [M−H]⁻.

### Example 5

### Hexyl 2-(2-cyano-4-nitrophenyl)-2-(2,4-dinitrophenyl)acetate

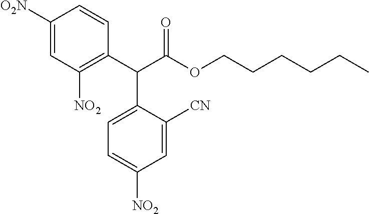 [SMILES: CCCCCCOC(=O)C(c1ccc([N+](=O)[O-])cc1C#N)c1ccc([N+](=O)[O-])cc1[N+](=O)[O-]]

### Step 1. Synthesis of Hexyl 2-(2,4-dinitrophenyl)acetate

See Example 1, Step 1.

### Step 2. Synthesis of Hexyl 2-(2-cyano-4-nitrophenyl)-2-(2,4-dinitrophenyl)acetate

A solution of hexyl 2-(2,4-dinitrophenyl)acetate (455 mg, 1.47 mmol) and 2-chloro-5-nitrobenzonitrile (402 mg, 2.20 mmol) in dry DMF (7 mL) was treated with anhydrous triethylamine (0.82 mL, 5.87 mmol). The reaction flask was immersed in a 60° C. oil bath and allowed to stir overnight under N₂. The reaction mixture was diluted with ethyl acetate (40 mL) and hexanes (10 mL). It was then treated dropwise with 1M hydrochloric acid until pH=1.0. The organic layer was separated, washed twice with water (2×50 mL) and washed with saturated aqueous NaCl (50 mL). The organic layer was dried over a mixture of Na₂SO₄ (4 g) and silica gel (4 g). The solids were removed by filtration, rinsing with dichloromethane. The combined filtrate and rinse were concentrated to an oil. The crude product was purified by chromatography (30 g SiO₂), eluting with dichloromethane to give 378 mg (65%) of a slightly yellow gum: MS ES-API m/z=455 [M−H]⁻.

### Example 6

### Stability of Hexyl 2,2-bis(2,4-dinitrophenyl)acetate to Reduction by dithiothreitol (DTT)

Dithiothreitol (DTT) is the common name for a small-molecule redox reagent known as Cleland's reagent. See Cleland, W. W. (1964). “Dithiothreitol, a new protective reagent for SH groups”. Biochemistry 3: 480-482. DTT is an unusually strong reducing agent, because once oxidized, it forms a stable six-membered ring with an internal disulfide bond. DTT has a redox potential of −0.33 V at pH 7.0. See M. J. O'Neil, ed. by (2001). Merck Index: an encyclopedia of chemicals, drugs, & biologicals: 13th ed. (13. ed. ed.). United States: MERCK & CO INC. The stability of Hexyl 2,2-bis(2,4-dinitrophenyl)acetate to reduction by DTT was qualitatively evaluated as follows:

A stock solution of Hexyl 2,2-bis(2,4-dinitrophenyl)acetate (from Example 1, 2.2 mg, 0.0046 mmol) in methanol (3.68 mL) was treated with 1×PCR buffer (0.92 mL, freshly prepared from Thermo Fischer 10×TAQ buffer with (NH₄)₂SO₄ and 20 mM MgCl₂). A visible spectrum of this solution was obtained using the Spectronic Genesis5 UV-Vis spectrophotometer and a 1 cm quartz cuvette (observed λ max=655 nM, Absorbance=3.205). The solution was subsequently treated with dithiothreitol (7.1 mg, 0.46 mmol, Aldrich Chemical). A visible spectrm of this solution was obtained using the Spectronic Genesis5 UV-Vis spectrophotometer and a 1 cm quartz cuvette (observed A max=655 nM, Absorbance=3.193). The solution was then placed in a sealed tube and heated in a 95 OC water bath for a period of 5 minutes. The solution was cooled to room temperature and a visible spectrum of this solution was observed to be essentially unchanged (λ max=655 nM, Absorbance=3.044).

1. A compound of formula (I) 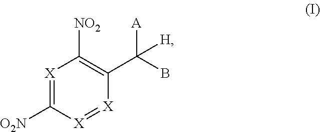
or a salt thereof, wherein:
A is CO₂R¹, CO₂R², C(O)NR³R⁴, CN, S(O)R⁵, SO₂R⁵, C(O)R⁶, C(O)R⁷, or CO(NHS);
B is selected from the group consisting of B¹, B², B³, and B⁴; wherein
B¹ is 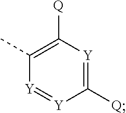
B² is selected from the group consisting of 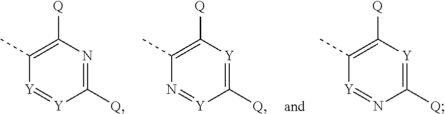
B³ is selected from the group consisting of 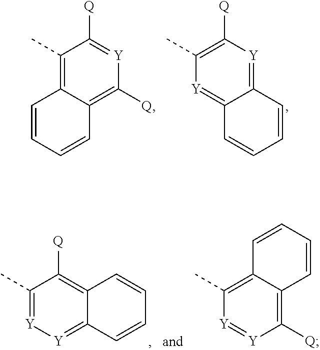
and
B⁴ is selected from the group consisting of 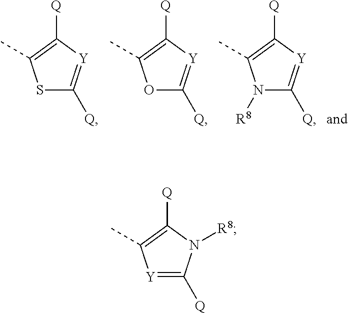
each X is independently N, C—H, C—Z¹, or C—Z²;
each Q is independently H, NO₂, Cl, Br, F, I, CH₃, OCH₃, CN, CO₂R⁹, C(O)NR¹⁰R¹¹, SO₂R¹², SOR¹², CF₃ or N₃;
R¹ is H or (C₁-C₁₂)alkyl;
R² is CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂OCH₂C≡CH, (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);
R³ is H, CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂OCH₂C≡CH, (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);
R⁴ is H, (C₁-C₆)alkyl, CH₂CH₂(C₄-C₁₂)perfluoroalkyl, or (CH₂CH₂O)ₙCH₂CH₂O(C₁-C₄)alkyl, or R³ and R⁴ together with the nitrogen atom to which they are attached form a ring selected from the group consisting of 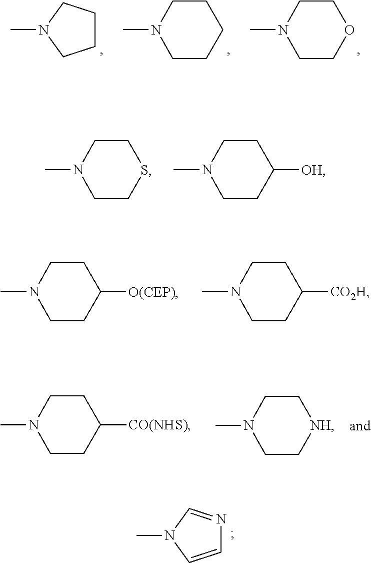
R⁵ is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;
R⁶ is H, (C₁-C₆)alkyl, or phenyl;
R⁷ is phenyl, wherein said phenyl is substituted with one to five substituents selected from the group consisting of CH₃, F, Cl, Br, I, OCH₃, OH, NO₂, CN, CF₃, N₃, and N(CH₃)₂;
R⁸ is H, (C₁-C₆)alkyl, CH₂CH₂(C₄-C₁₂)perfluoroalkyl), or (CH₂CH₂O)ₙCH₂CH₂O(C₁-C₄)alkyl;
R⁹ is H, CH₂CH₂(C₄-C₁₂)perfluoroalkyl), or (CH₂CH₂O)ₙCH₂CH₂O(C₁-C₄)alkyl;
R¹⁰ is H, CH₂CH₂(C₄-C₁₂)perfluoroalkyl), or (CH₂CH₂O)ₙCH₂CH₂O(C₁-C₄)alkyl;
R¹¹ is H, (C₁-C₆)alkyl, or (CH₂CH₂O)ₙCH₂CH₂O(C₁-C₄)alkyl, or R¹⁰ and R¹¹ together with the nitrogen atom to which they are attached form a ring selected from the group consisting of 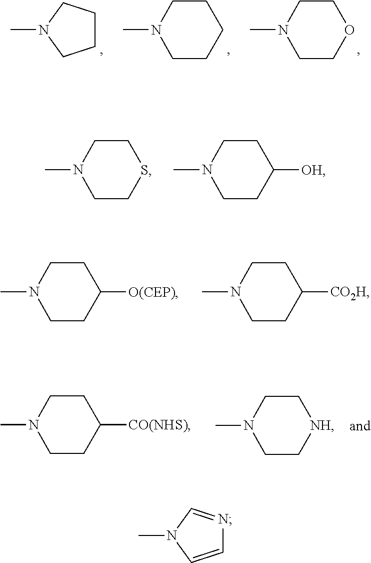
R¹² is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;
each Y is independently N, C—H, C—Z¹, or C—Z²;
each Z¹ is independently OCH₃, CN, or CF₃;
each Z² is independently CH₃, F, Cl, Br, or I;
m is an integer from 2 to 8; and
each n is independently an integer from 1 to 5;
wherein if each X is independently C—H or C—Z², and A is CO₂R¹, CN, or COR^{E}, then B is B², B³, or B⁴.

2. The compound or salt of claim 1, wherein each
X is C—H, A is CO₂R¹, and B is B², B³, or B⁴;
each X is C—H, A is CN, and B is B², B³, or B⁴;
each X is C—H, A is COR⁶, and B is B², B³, or B⁴;
at least one X is N or C—Z¹ and A is CO₂R¹, CN or COR⁶; or
A is CO₂R², C(O)NR³R⁴, S(O)R⁵, SO₂R⁵, C(O)R⁷, or CO(NHS).

3. (canceled)

4. (canceled)

5. (canceled)

6. (canceled)

7. (canceled)

8. (canceled)

9. (canceled)

10. The compound or salt of claim 1, wherein A is CO₂R¹ and R¹ is (C₁-C₁₂)alkyl;
A is CO₂R² and R² is CH₂CH₂(C₄-C₁₂)perfluoroalkyl or (CH₂CH₂O)ₙCH₂CH₂O(C₁-C₄)alkyl;
A is CO₂R² and R² is (CH₂) OH, (CH₂)ₘSH, (CH₂)ₘNH₂, (CH)ₘCO(NHS) or —(CH₂)ₘ(N-maleimide);
A is CO₂R² and R² is —(CH₂CH₂O)ₙCH₂CH₂OH, —(CH₂CH₂O)ₙCH₂CH₂SH, —(CH₂CH₂O)ₙCH₂CH₂NH₂, —(CH₂CH₂O)ₙCH₂CH₂CO(NHS) or —(CH₂CH₂O)ₙCH₂CH₂(N-maleimide);
A is CO₂R² and R² is —(CH₂)ₘN₃, —(CH₂CH₂O)CH₂CH₂N₃, —(CH₂)ₘOCH₂C≡CH or —(CH₂CH₂O)ₙCH₂CH₂OCH₂C≡CH;
A is CO₂R² and R² is —(CH₂)ₘO(CEP), —(CH₂CH₂O)ₙCH₂CH₂O(CEP), —(CH₂)₃OCH₂CH(OH)CH₂OH, —(CH₂CH₂O)ₙCH₂CH(OH)CH₂OH, —CH₂)₃OCH₂CH(OH)CH₂O(DMT), —(CH₂CH₂O)ₙCH₂CH(OH)CH₂O(DMT), —(CH₂)₃OCH₂CH(O(CEP))CH₂O(DMT) or —(CH₂CH₂O)ₙCH₂CH(O(CEP))CH₂O(DMT);
A is C(O)NR³R⁴ and R³ is (C₁-C₁₂)alkyl;
A is C(O)NR³R⁴ and R³ is (CH₂)ₘOH, (CH₂)ₘSH, (CH₂)ₘNH₂, (CH₂)ₘCO(NHS) or —(CH₂)ₘ(N-maleimide);
A is C(O)NR³R⁴ and R³ is —(CH₂CH₂O)ₙCH₂CH₂OH, —(CH₂CH₂O)ₙCH₂CH₂SH, —(CH₂CH₂O)ₙCH₂CH₂NH₂, —(CH₂CH₂O)ₙCH₂CH₂CO(NHS) or —(CH₂CH₂O)ₙCH₂CH₂(N-maleimide);
A is C(O)NR³R⁴ and R³ is (CH₂)ₘN₃, —(CH₂CH₂O)ₙCH₂CH₂N₃, —(CH₂)ₘOCH₂C≡CH or —(CH₂CH₂O)ₙCH₂CH₂OCH₂C≡CH;
A is C(O)NR³R⁴ and R³ is —(CH₂)ₘO(CEP), —(CH₂CH₂O)ₙCH₂CH₂O(CEP), —(CH₂)₃OCH₂CH(OH)CH₂OH, —(CH₂CH₂O)ₙCH₂CH(OH)CH₂OH, —(CH₂)₃OCH₂CH(OH)CH₂O(DMT), —(CH₂CH₂O)ₙCH₂CH(OH)CH₂O(DMT), —(CH₂)₃OCH₂CH(O(CEP))CH₂O(DMT) or —(CH₂CH₂O)ₙCH₂CH(O(CEP))CH₂O(DMT); or
A is C(O)NR³R⁴ and R³ and R⁴ together with the nitrogen atom to which they are attached form a ring selected from the group consisting of

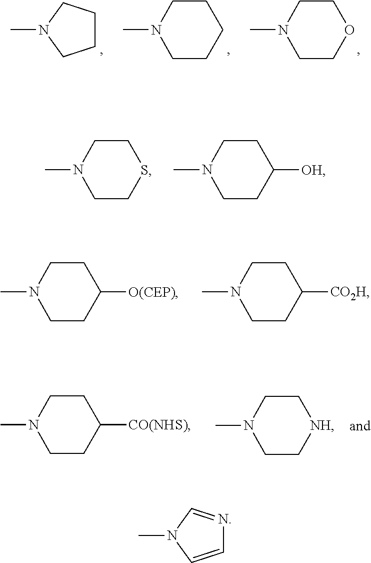

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The compound or salt of claim 1, wherein the compound of formula (I) is a compound of formula (Ia) 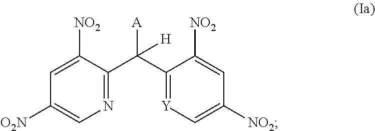
a compound of formula (Ib) 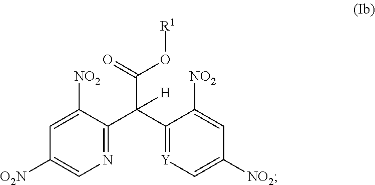
a compound of formula (Ic) 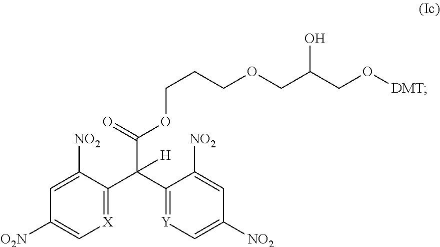
a compound of formula (Id) 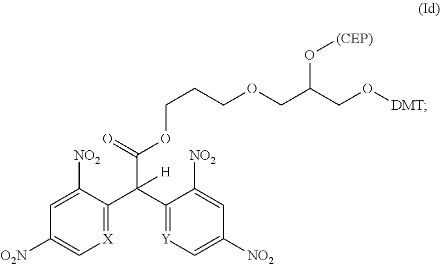
a compound of formula (Ie) 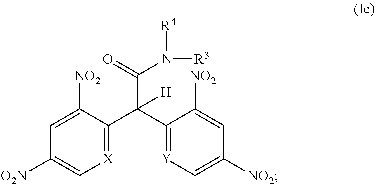
a compound of formula (If) 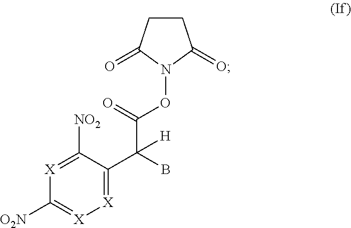
or
a compound of formula (Ig)

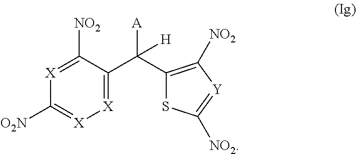

23. (canceled)

24. (canceled)

25. (canceled)

26. (canceled)

27. (canceled)

28. (canceled)

29. The compound or salt of claim 1, wherein each X is C—H.

30. The compound or salt of claim 1, wherein each Y is C—H.

31. The compound or salt of claim 1, wherein at least one occurrence of X is N.

32. The compound or salt of claim 1, wherein at least one occurrence of Y is N.

33. (canceled)

34. (canceled)

35. (canceled)

36. (canceled)

37. The compound or salt of claim 1, wherein the compound or salt is selected from the group consisting of: 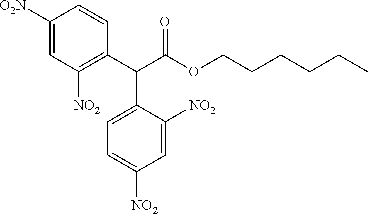 [SMILES: CCCCCCOC(=O)C(c1ccc([N+](=O)[O-])cc1[N+](=O)[O-])c1ccc([N+](=O)[O-])cc1[N+](=O)[O-]]
Hexyl 2,2-bis(2,4-dinitrophenyl)acetate; 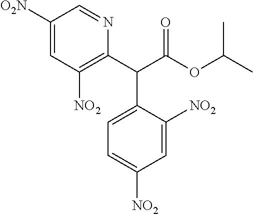 [SMILES: CC(C)OC(=O)C(c1ccc([N+](=O)[O-])cc1[N+](=O)[O-])c1ncc([N+](=O)[O-])cc1[N+](=O)[O-]]
Isopropyl 2-(2,4-dinitrophenyl)-2-(3,5-dinitropyridin-2-yl)acetate; 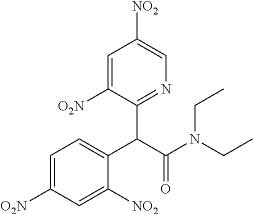 [SMILES: CCN(CC)C(=O)C(c1ccc([N+](=O)[O-])cc1[N+](=O)[O-])c1ncc([N+](=O)[O-])cc1[N+](=O)[O-]]
2-(2,4-dinitrophenyl)-2-(3,5-dinitropyridin-2-yl)-N, N-diethylacetamide; 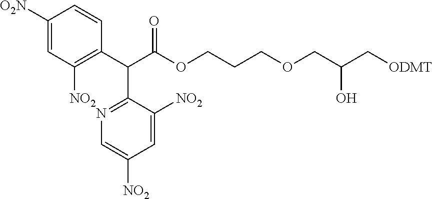
3-(3-(bis(4-methoxyphenyl)(phenyl)methoxy)-2-hydroxypropoxy)propyl 2-(2,4-dinitrophenyl)-2-(3,5-dinitropyridin-2-yl)acetate; 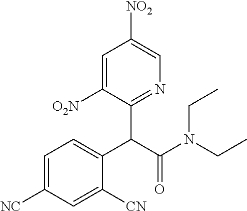 [SMILES: CCN(CC)C(=O)C(c1ccc(C#N)cc1C#N)c1ncc([N+](=O)[O-])cc1[N+](=O)[O-]]
2-(2,4-dicyanophenyl)-2-(3,5-dinitropyridin-2-yl)-N,N-diethylacetamide; 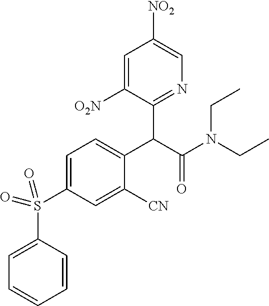 [SMILES: CCN(CC)C(=O)C(c1ccc(S(=O)(=O)c2ccccc2)cc1C#N)c1ncc([N+](=O)[O-])cc1[N+](=O)[O-]]
2-(3,5-dinitropyridin-2-yl)-N,N-diethyl-2-(4-(phenylsulfonyl)phenyl)acetamide; 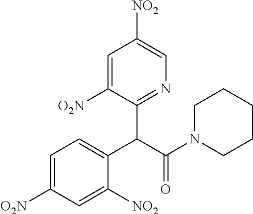 [SMILES: O=C(C(c1ccc([N+](=O)[O-])cc1[N+](=O)[O-])c1ncc([N+](=O)[O-])cc1[N+](=O)[O-])N1CCCCC1]
2-(2,4-dinitrophenyl)-2-(3,5-dinitropyridin-2-yl)-1-(piperidin-1-yl)ethan-1-one; 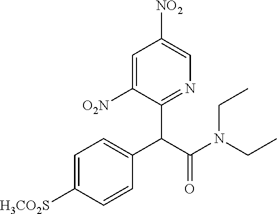
2-(3,5-dinitropyridin-2-yl)-N,N-diethyl-2-(4-(methylsulfonyl)phenyl)acetamide; 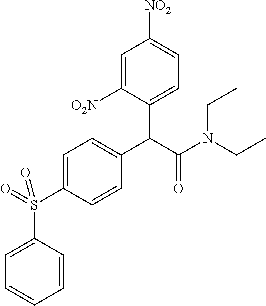 [SMILES: CCN(CC)C(=O)C(c1ccc(S(=O)(=O)c2ccccc2)cc1)c1ccc([N+](=O)[O-])cc1[N+](=O)[O-]]
2-(2,4-dinitrophenyl)-N,N-diethyl-2-(4-(phenylsulfonyl)phenyl)acetamide; and 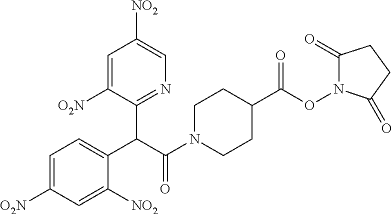 [SMILES: O=C(ON1C(=O)CCC1=O)C1CCN(C(=O)C(c2ccc([N+](=O)[O-])cc2[N+](=O)[O-])c2ncc([N+](=O)[O-])cc2[N+](=O)[O-])CC1]
2,5-dioxopyrrolidin-1-yl 1-(2-(2,4-dinitrophenyl)-2-(3,5-dinitropyridin-2-yl)acetyl)piperidine-4-carboxylate;
or a salt thereof.

38. The compound or salt of claim 1, wherein the compound or salt is: 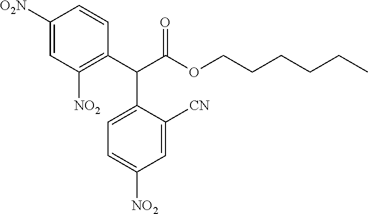 [SMILES: CCCCCCOC(=O)C(c1ccc([N+](=O)[O-])cc1C#N)c1ccc([N+](=O)[O-])cc1[N+](=O)[O-]]
Hexyl 2-(2-cyano-4-nitrophenyl)-2-(2,4-dinitrophenyl)acetate;
or a salt thereof.

39. The compound or salt of claim 1, wherein:
A is CO₂R¹, CO₂R², C(O)NR³R⁴, CN, S(O)R⁵, SO₂R⁵, C(O)R⁶, or C(O)R⁷;
R² is CH₂CH₂(C₄-C₁₂)perfluoroalkyl;
R³ is H, (C₁-C₁₂)alkyl, or CH₂CH₂(C₄-C₁₂)perfluoroalkyl; and
R⁴ is H, (C₁-C₆)alkyl, CH₂CH₂(C₄-C₁₂)perfluoroalkyl, or (CH₂CH₂O)ₙCH₂CH₂O(C₁-C₄)alkyl, or R³ and R⁴ together with the nitrogen atom to which they are attached form a ring selected from the group consisting of

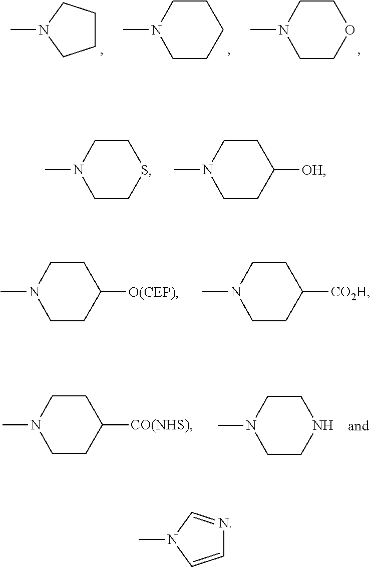

40. A covalent conjugate of formula (II) 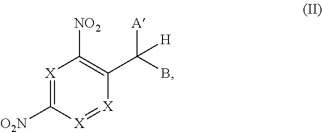
or a salt thereof, wherein,
A′ is CO₂R¹, CO₂R^{2′}, C(O)NR^{3′}R^{4′}, CN, S(O)R⁵, SO₂R⁵, C(O)R⁶, C(O)R⁷, CO(NHS), or CO-(L-[Conjugated Species]);
B is selected from the group consisting of B¹, B², B³, and B⁴; wherein
B¹ is 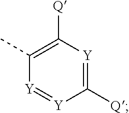
B² is selected from the group consisting of 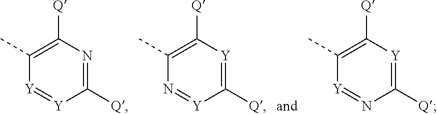
B³ is selected from the group consisting of 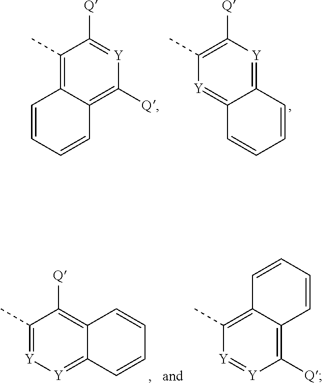
and
B⁴ is selected from the croup consisting of 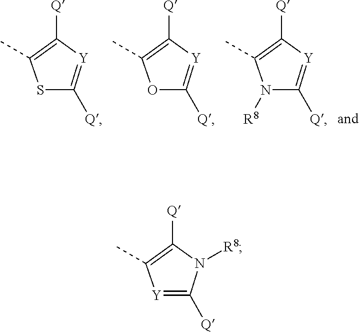
each X is independently N, C—H, C—Z¹, or C—Z²;
L is a single covalent bond or a covalent linkage having 1-50 non-hydrogen atoms selected from the group consisting of C, N, O, S and P and is composed of any combination of single, double, triple or aromatic bonds;
each Q′ is independently H, NO₂, Cl, Br, F, I, CH₃, OCH₃, CN, CO₂R⁹, C(O)NR^{1′}R^{11′}, SO₂R¹², SOR¹², CF₃, N₃, or CO-(L-[Conjugated Species]);
R¹ is H or (C₁-C₁₂)alkyl;
R^{2′} is CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂)ₘ-L-[Conjugated Species], (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂-L-[Conjugated Species], (CH₂CH₂O)ₙCH₂CH₂OCH₂C≡CH, (CH₂CH₂O)ₙCH₂-L-[Conjugated Species], (CH₂)₃OCH₂CH(OH)CH₂OH, (CH₂)₃OCH₂CH(OH)CH₂O(DMT), (CH₂)₃OCH₂CH(O(CEP))CH₂O(DMT), (CH₂)₃OCH₂CH(L-[Conjugated Species])CH₂OH, (CH₂)₃OCH₂CH(L-[Conjugated Species])CH₂-L-[Conjugated Species], (CH₂CH₂)ₙOCH₂CH(OH)CH₂OH, (CH₂CH₂)ₙOCH₂CH(OH)CH₂O(DMT), (CH₂CH₂)ₙOCH₂CH(O(CEP))CH₂O(DMT), (CH₂CH₂)ₙOCH₂CH(L-[Conjugated Species])CH₂OH, or (CH₂CH₂)ₙOCH₂CH(L-[Conjugated Species])CH₂-L-[Conjugated Species];
R^{3′} is H, (C₁-C₁₂)alkyl, CH₂CH₂(C₄-C₁₂)perfluoroalkyl, (CH₂)ₘOH, (CH₂)ₘSH, (CH₂)ₘN₃, (CH₂)ₘNH₂, (CH₂)ₘCO(NHS), (CH₂)ₘ(N-maleimide), (CH₂)ₘO(CEP), (CH₂)ₘOCH₂C≡CH, (CH₂)ₘ-L-[Conjugated Species], (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂CH₂O)ₙCH₂CH₂-L-[Conjugated Species], (CH₂CH₂O)ₙCH₂CH₂OCH₂C≡CH, (CH₂CH₂O)ₙCH₂-L-[Conjugated Species], (CH₂)₃OCH₂CH(OH)CH₂OH, (CH₂)₃OCH₂CH(OH)CH₂O(DMT), (CH₂)₃OCH₂CH(O(CEP))CH₂O(DMT), (CH₂)₃OCH₂CH(L-[Conjugated Species])CH₂OH, (CH₂)₃OCH₂CH(L-[Conjugated Species])CH₂-L-[Conjugated Species], (CH₂CH₂)ₙOCH₂CH(OH)CH₂OH, (CH₂CH₂)ₙOCH₂CH(OH)CH₂O(DMT), (CH₂CH₂)ₙOCH₂CH(O(CEP))CH₂O(DMT), (CH₂CH₂O)ₙOCH₂CH(L-[Conjugated Species])CH₂OH, or (CH₂CH₂O)ₙOCH₂CH(L-[Conjugated Species])CH₂-L-[Conjugated Species];
R^{4′} is H, (C₁-C₆)alkyl, CH₂CH₂(C₄-C₁₂)perfluoroalkyl, or (CH₂CH₂O)ₙCH₂CH₂O(C₁-C₄)alkyl, or R^{3′} and R^{4′} together with the nitrogen atom to which they are attached form a ring selected from the group consisting of 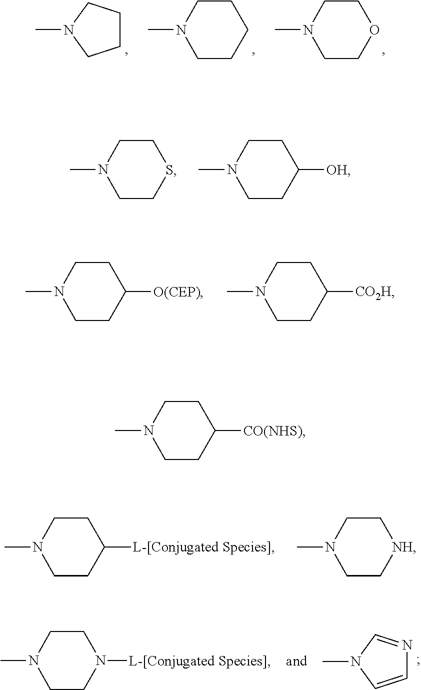
R⁵ is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;
R⁶ is H, (C₁-C₆)alkyl, or phenyl;
R⁷ is phenyl, wherein said phenyl is substituted with one to five substituents selected from the group consisting of CH₃, F, Cl, Br, I, OCH₃, OH, NO₂, CN, CF₃, N₃, and N(CH₃)₂;
Fe is H, (C₁-C₆)alkyl, CH₂CH₂(C₄-C₁₂)perfluoroalkyl), or (CH₂CH₂O)ₙCH₂CH₂O(C₁-C₄)alkyl;
R⁹ is H, (C₁-C₁₂)alkyl, CH₂CH₂(C₄-C₁₂)perfluoroalkyl), or (CH₂CH₂O)ₙCH₂CH₂O(C₁-C₄)alkyl;
R^{10′} is H, CH₂CH₂(C₄-C₁₂)perfluoroalkyl), or (CH₂CH₂O)ₙCH₂CH₂O(C₁-C₄)alkyl;
R^{11′} is H, (C₁-C₆)alkyl, or (CH₂CH₂O)ₙCH₂CH₂O(C₁-C₄)alkyl, or R^{10′} and R^{11′} together with the nitrogen atom to which they are attached form a ring selected from the group consisting of 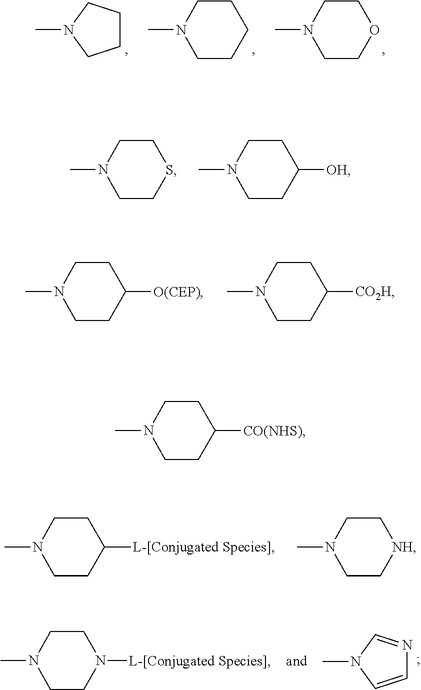
R¹² is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;
each Y is independently N, C—H, C—Z¹, or C—Z²;
each Z¹ is independently OCH₃, CN, or CF₃;
each Z² is independently CH₃, F, Cl, Br, or I;
m is an integer from 2 to 8; and
each n is independently an integer from 1 to 5;
wherein the covalent conjugate of formula (II) includes at least one occurrence of -L-[Conjugated Species].

41. The covalent conjugate of claim 40, wherein each X is C—H.

42. The covalent conjugate of claim 40, wherein each Y is C—H.

43. The covalent conjugate of claim 40, wherein at least one occurrence of X is N.

44. The covalent conjugate of claim 40, wherein at least one occurrence of Y is N.

45. (canceled)

46. (canceled)

47. (canceled)

48. (canceled)

49. The covalent conjugate or salt of any one of claim 40, wherein the Conjugated Species is a protein, an antibody, a peptide, a nucleic acid, an oligonucleotide, a solid support, a soluble polymer, an insoluble polymer, a dendrimer, a saccharide, a fatty acid, a lipid, or a phospholipid.

50. (canceled)

51. (canceled)

52. (canceled)

53. (canceled)

54. (canceled)

55. (canceled)

56. A method for detecting a change in the pH of a mixture, comprising:
providing a mixture comprising a fluorescence donor and the compound or salt of claim 1;
irradiating the resulting mixture at a first time with light having a wavelength suitable to excite the fluorescence donor;
measuring detectable fluorescence emitted by the fluorescence donor while irradiating the mixture at said first time;
irradiating the resulting mixture at a second time with light having a wavelength suitable to excite the fluorescence donor;
measuring detectable fluorescence emitted by the fluorescence donor while irradiating the mixture at said second time; and
comparing the detectable fluorescence emitted by the fluorescence donor at said first time with the detectable fluorescence emitted by the fluorescence donor at said second time to detect a change in the pH of the mixture;
wherein a decrease in detectable fluorescence at said second time relative to said first time indicates an increase in the pH of the mixture, and wherein an increase in detectable fluorescence at said second time relative to said first time indicates a decrease in the pH of the mixture.

57. A method for detecting a change in the pH of a mixture, comprising:
providing a mixture comprising a fluorescence donor and a compound of formula (I) 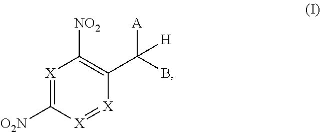
or a salt thereof, wherein:
A is CO₂R¹, CO₂R², C(O)NR³R⁴, CN, S(O)R⁵, SO₂R⁵, C(O)R⁶, C(O)R⁷, or CO(NHS);
B is selected from the group consisting of B¹, B², B³, and B⁴; wherein
B¹ is 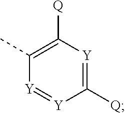
B² is selected from the group consisting of 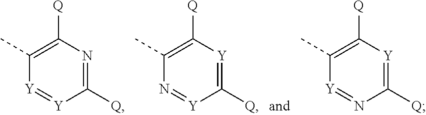
B³ is selected from the group consisting of 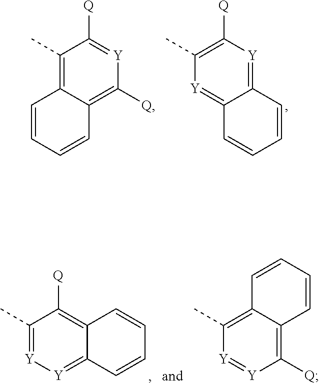
and
B⁴ is selected from the group consisting of 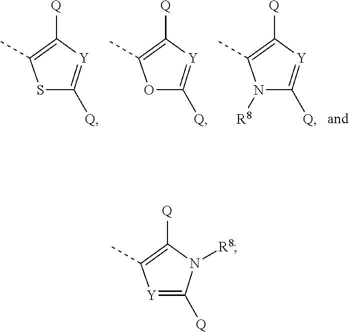
each X is independently N, C—H, C—Z¹, or C—Z²;
each Q is independently H, NO₂, Cl, Br, F, I, CH₃, OCH₃, CN, CO₂R⁹, C(O)NR¹⁰R¹¹, SO₂R¹², SOR¹², CF₃ or N₃;
R¹ is H or (C₁-C₁₂)alkyl;
R² is CH₂CH₂(C₄-C₁₂)perfluoroalkyl, (CH₂)ₘOH, (CH₂)ₘSH, (CH₂)ₘN₃, (CH₂)ₘOCH₂C≡CH, (CH₂)ₘNH₂, (CH₂)ₘCO(NHS), (CH₂)ₘ(N-maleimide), (CH₂)ₘO(CEP), (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂OCH₂C≡CH, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);
R³ is H, CH₂CH₂(C₄-C₁₂)perfluoroalkyl, (CH₂)ₘOH, (CH₂)ₘSH, (CH₂)ₘN₃, (CH₂)ₘOCH₂C≡CH, (CH₂)ₘNH₂, (CH₂)ₘCO(NHS), (CH₂)ₘ(N-maleimide), (CH₂)ₘO(CEP), (CH₂CH₂O)ₙCH₂CH₂O(C₁-C₄)alkyl, (CH₂CH₂O)ₙCH₂CH₂OH, (CH₂CH₂O)ₙCH₂CH₂SH, (CH₂CH₂O)ₙCH₂CH₂N₃, (CH₂CH₂O)ₙCH₂CH₂OCH₂C≡CH, (CH₂CH₂O)ₙCH₂CH₂NH₂, (CH₂CH₂O)ₙCH₂CH₂CO(NHS), (CH₂CH₂O)ₙCH₂CH₂(N-maleimide), (CH₂CH₂O)ₙCH₂CH₂O(CEP), (CH₂)₃OCH₂CH(OH)CH₂OH, (CH₂CH₂)ₙOCH₂CH(OH)CH₂OH, (CH₂)₃OCH₂CH(OH)CH₂O(DMT), (CH₂CH₂)ₙOCH₂CH(OH)CH₂O(DMT), (CH₂)₃OCH₂CH(O(CEP))CH₂O(DMT) or (CH₂CH₂)ₙOCH₂CH(O(CEP))CH₂O(DMT);
R⁴ is H, (C₁-C₆)alkyl, CH₂CH₂(C₄-C₁₂)perfluoroalkyl, or (CH₂CH₂O)ₙCH₂CH₂O(C₁-C₄)alkyl, or R³ and R⁴ together with the nitrogen atom to which they are attached form a ring selected from the group consisting of 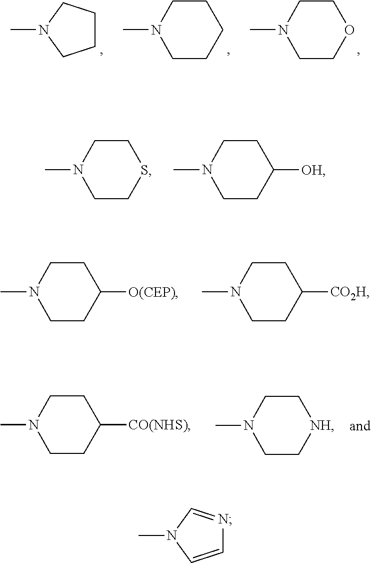
R⁵ is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;
R⁶ is H, (C₁-C₆)alkyl, or phenyl;
R⁷ is phenyl, wherein said phenyl is substituted with one to five substituents selected from the group consisting of CH₃, F, Cl, Br, I, OCH₃, OH, NO₂, CN, CF₃, N₃, and N(CH₃)₂;
R⁸ is H, (C₁-C₆)alkyl, CH₂CH₂(C₄-C₁₂)perfluoroalkyl), or (CH₂CH₂O)ₙCH₂CH₂O(C₁-C₄)alkyl;
R⁹ is H, CH₂CH₂(C₄-C₁₂)perfluoroalkyl), or (CH₂CH₂O)ₙCH₂CH₂O(C₁-C₄)alkyl;
R¹⁰ is H, CH₂CH₂(C₄-C₁₂)perfluoroalkyl), or (CH₂CH₂O)ₙCH₂CH₂O(C₁-C₄)alkyl;
R¹¹ is H, (C₁-C₆)alkyl, or (CH₂CH₂O)ₙCH₂CH₂O(C₁-C₄)alkyl, or R¹⁰ and R¹¹ together with the nitrogen atom to which they are attached form a ring selected from the group consisting of 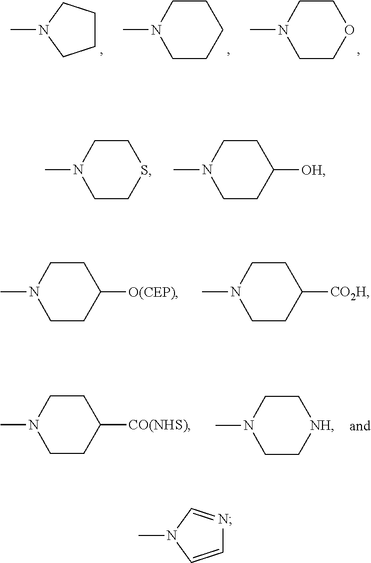
R¹² is (C₁-C₆)alkyl or phenyl, wherein said phenyl may be unsubstituted or substituted with up to five substituents selected from the group consisting of CH₃, F, Cl, Br, I, OCH₃, OH, NO₂, CN, CF₃, N₃, and N(CH₃)₂;
each Y is independently N, C—H, C—Z¹, or C—Z²;
each Z¹ is independently OCH₃, CN, or CF₃;
each Z² is independently CH₃, F, Cl, Br, or I;
m is an integer from 2 to 8;
and
each n is independently an integer from 1 to 5;
irradiating the resulting mixture at a first time with light having a wavelength suitable to excite the fluorescence donor;
measuring detectable fluorescence emitted by the fluorescence donor while irradiating the mixture at said first time;
irradiating the resulting mixture at a second time with light having a wavelength suitable to excite the fluorescence donor; and
measuring detectable fluorescence emitted by the fluorescence donor while irradiating the mixture at said second time; and
comparing the detectable fluorescence emitted by the fluorescence donor at said first time with the detectable fluorescence emitted by the fluorescence donor at said second time to detect a change in the pH of the mixture;
wherein a decrease in detectable fluorescence at said second time relative to said first time indicates an increase in the pH of the mixture, and wherein an increase in detectable fluorescence at said second time relative to said first time indicates a decrease in the pH of the mixture.

58. A method for detecting a change in the pH of a mixture, comprising:
providing a mixture comprising a fluorescence donor and the covalent conjugate or salt of claim 40;
irradiating the resulting mixture at a first time with light having a wavelength suitable to excite the fluorescence donor;
measuring detectable fluorescence emitted by the fluorescence donor while irradiating the mixture at said first time;
irradiating the resulting mixture at a second time with light having a wavelength suitable to excite the fluorescence donor;
measuring detectable fluorescence emitted by the fluorescence donor while irradiating the mixture at said second time; and
comparing the detectable fluorescence emitted by the fluorescence donor at said first time with the detectable fluorescence emitted by the fluorescence donor at said second time to detect a change in the pH of the mixture;
wherein a decrease in detectable fluorescence at said second time relative to said first time indicates an increase in the pH of the mixture, and wherein an increase in detectable fluorescence at said second time relative to said first time indicates a decrease in the pH of the mixture.
